SEQUENCE LISTING (50 sequences)

SEQ ID NO: 1 - protein, 772 aa, Guillardia theta
MDSTEATAASRTWCNFETSRTILFIFGLIQSFSCSGIIFGFTAIASALKNIGQYSSLCSSTNSTSGTVHCANQDLRFSLLYTIGSSALFASFICHGIILDRIGPKIASCTGLTCISIGFYILSFSDSNGFDGYPEAFALIGRGPGVHLSLFHLANLFREGIRGTMKSCLVGAFVMSAILFPLEGWLLEGVHANREYVFLVHASWLMLLALAGAFVWPIKRFQDGDKVYLRGFLLRYTIVPHSSIVENVEMPPQVTEDVVLEASKSEVISEYTSKSDGTQGDIDTQTDLGTESNARIPGTASNSLVNAPLLRQMRSKEYLLLFSFACIHVLRFNLYIGTFIQQIQYLEGKGNSSQTNVYTLALTLLLPFGALSIPLIGRIIDRYSLWHGFIFCNMLGMGYGITNVVPVLQVQFLSIIFISLHRMVLFAVLYSYTSATFGYANFGFLFGLMQLVAAVISLLVYPLASISDNQLGGNYMCVDLVMLAIMMPLWYFCPGQRPQGTASLNRSPTKMRGFRSMFAKQATALFIGGAALAGGSVAWAEGYKDAHSVAGEWRLPRPHYEVLVSSCFTAICLAVSLYLYVPKLLVPKIGTIIKNYFFPPTLKLPEWEEEIASRPGLKLQAQVSRFAQSSTDGALLVCMPRIYIPETPYSEEQEEEDKKNQKGYESRDVYCTWHFGKDAVGHPEIVHGGAIATAIDESCGHAFMALGRGLGFTAYLHVNYKKPTPAGIPLLITVKLKSIEGRKVTLDAVVSDGKQTEFADGSALFVVPRKKE

SEQ ID NO: 2 - DNA, 2319 bp, Guillardia theta
atggattcaacggaagcgaccgctgcttccagaacctggtgcaacttcgaaacttcccgcaccattctcttcattttcggcctgattcaatcattcagctgctcgggaatcatatttggtttcacagccattgctagcgctctgaaaaacattggccagtactcttctctatgcagctccaccaattcaacgtcaggcacggtacattgcgccaatcaagacctgcggtttagccttctgtataccatcggatcatctgcgttgttcgcttccttcatctgccatggcataattctcgaccgcattgggcctaaaattgcatcatgcacaggtttgacctgcatatctatcgggttttacatcttgtcgtttagcgactccaacggcttcgatggatacccagaagcgttcgccttgatcgggcggggccccggcgtgcatctttctctgtttcaccttgccaacttattcagagaggggatcagagggacaatgaagagctgcctggtcggtgcgtttgtcatgagcgcgattctctttcctctcgagggctggttgctggagggcgtgcatgcgaacagggagtacgtcttcctcgtgcatgcctcctggttgatgcttctcgcccttgctggagcatttgtctggcccatcaaacggtttcaagacggagacaaggtctatctaagggggtttcttctgagatacaccatcgttccgcacagcagcattgtcgaaaatgtcgagatgcccccacaagtgaccgaggacgtcgttctcgaagcatccaagtctgaagtcatctctgagtatacctctaagtccgatggaactcaaggcgatatagacacgcagactgatctcggtactgaatccaatgctcgtatccccgggacagcctcgaattctttggtgaatgcccctctgttacgccagatgcgatcgaaagagtacctgctgctcttcagctttgcttgtattcatgtcctacgcttcaacttgtacattggcactttcatacagcagatacagtatcttgaaggaaaaggcaattcttcacaaacaaatgtttatactctggcactaacacttcttctaccttttggtgccttgtctatacctttaatcgggcgtataattgaccgatacagcctttggcacggtttcatattttgcaatatgctgggaatgggctacggtattacaaatgtcgtgcctgtgcttcaggtgcagtttttgtccatcatcttcatcagccttcaccgtatggtcctctttgcagtgctctactcttacacctcagccacgttcgggtatgccaacttcgggtttctcttcggactcatgcagctagtggctgcggtcatttctctcttggtctatcccctcgctagtatctccgacaaccagctaggcgggaactacatgtgtgtcgaccttgtgatgcttgcaataatgatgcccttgtggtacttttgcccgggtcaacgacctcaaggcactgcaagcctgaacaggagcccgacaaaaatgcggggattcagaagcatgtttgcaaagcaagctactgcgctcttcatcggcggagcggctcttgcaggaggaagcgtggcatgggcagagggatacaaggatgcgcattcggtagcgggagaatggaggctgcctcgacctcattacgaggtattggtctcttcatgcttcaccgccatctgtctggcagttagtttgtacctctatgtgccaaagctgctcgtgcccaagatcggaaccatcatcaagaactatttcttccctccgaccttgaagctcccagagtgggaggaagagatcgcttccaggcccgggttgaagttgcaggcccaggtgagtcggtttgctcagagcagcactgatggagctttgctggtctgcatgccgaggatttacatcccggaaactccgtacagtgaagagcaggaggaggaggacaagaagaaccagaagggatacgaatcaagagacgtgtactgcacatggcactttgggaaggatgcggtgggccaccccgagatcgttcacggaggagcgatagccaccgccatcgacgagtcttgcgggcacgcgttcatggcgttggggaggggacttggcttcacggcctacctgcatgtcaactacaagaagccgacccctgccgggattcctctactgatcacagtcaagctaaagagcattgaagggcgaaaggtaactctggacgctgttgtcagcgacggcaagcagacagaattcgctgacggatcggctctgtttgtggttccaagaaagaaggagtag

SEQ ID NO: 3 - DNA, 861 bp, Artificial Sequence
  GtTE gene
atgccgctgtggtacttttgtccggggcagcgtccgcaaggcactgcctcgttaaatcgcagcccaaccaaaatgcgcggcttccgctcaatgtttgccaaacaagcaacagccctcttcattggcggtgcagcattggcggggggttcagttgcctgggcggaaggctacaaagatgcccactcagtggcaggcgaatggcgattacctcgtccccactatgaggttctagtgtcgagctgtttcacggccatttgccttgcggttagcctgtatctctatgtcccgaaacttctggtcccgaaaatcggcaccatcatcaaaaactacttctttccgcccaccctgaagctcccggaatgggaggaggaaattgcgtctcgtcctggactgaagttgcaagcacaggtttctcgctttgcccagagctccaccgatggcgcgttactggtgtgcatgccgcgcatttatatcccggaaaccccctatagcgaagaacaggaggaagaggataagaagaatcagaaaggctacgaaagtcgtgatgtctattgcacgtggcattttggcaaagacgccgtaggtcacccggaaatagtgcatggtggggcgattgcgacagctattgacgaaagctgtggacatgccttcatggcgttaggtcgcggtcttggatttacggcatacctgcatgtgaactacaagaaaccaaccccagctggcattcctttgctgattacggtaaaactgaaatccatcgaaggtcgtaaagtgactctggatgctgtggtgagtgacggcaaacagaccgagtttgcggatggctcggcgctgttcgtagttccgcggaaaaaagaataa

SEQ ID NO: 4 - DNA, 35 bp, Artificial Sequence
  PCR primer 1 for GtTE
gcggccgctctagagccgctgtggtacttttgtcc

SEQ ID NO: 5 - DNA, 35 bp, Artificial Sequence
  PCR primer 2 for GtTE
acaaaatattaacgcttattcttttttccgcggaa

SEQ ID NO: 6 - DNA, 20 bp, Artificial Sequence
  PCR primer 1 for pBluescriptII
ctctagagcggccgccaccg

SEQ ID NO: 7 - DNA, 24 bp, Artificial Sequence
  PCR primer 2 for pBluescriptII
gcgttaatattttgttaaaattcg

SEQ ID NO: 8 - DNA, 35 bp, Artificial Sequence
  PCR primer 1 for GtTE_497
gcggccgctctagagcgtccgcaaggcactgcctc

SEQ ID NO: 9 - DNA, 35 bp, Artificial Sequence
  PCR primer 1 for GtTE_507
gcggccgctctagagagcccaaccaaaatgcgcgg

SEQ ID NO: 10 - DNA, 35 bp, Artificial Sequence
  PCR primer 1 for GtTE_517
gcggccgctctagagatgtttgccaaacaagcaac

SEQ ID NO: 11 - DNA, 35 bp, Artificial Sequence
  PCR primer 1 for GtTE_527
gcggccgctctagagattggcggtgcagcattggc

SEQ ID NO: 12 - DNA, 35 bp, Artificial Sequence
  PCR primer 1 for GtTE_537
gcggccgctctagaggttgcctgggcggaaggcta

SEQ ID NO: 13 - DNA, 35 bp, Artificial Sequence
  PCR primer 1 for GtTE_547
gcggccgctctagagcactcagtggcaggcgaatg

SEQ ID NO: 14 - DNA, 35 bp, Artificial Sequence
  PCR primer 1 for GtTE_557
gcggccgctctagagcgtccccactatgaggttct

SEQ ID NO: 15 - DNA, 35 bp, Artificial Sequence
  PCR primer 1 for GtTE_567
gcggccgctctagagtgtttcacggccatttgcct

SEQ ID NO: 16 - DNA, 35 bp, Artificial Sequence
  PCR primer 1 for GtTE_577
gcggccgctctagagctgtatctctatgtcccgaa

SEQ ID NO: 17 - DNA, 35 bp, Artificial Sequence
  PCR primer 1 for GtTE_587
gcggccgctctagagccgaaaatcggcaccatcat

SEQ ID NO: 18 - DNA, 35 bp, Artificial Sequence
  PCR primer 1 for GtTE_597
gcggccgctctagagttctttccgcccaccctgaa

SEQ ID NO: 19 - DNA, 35 bp, Artificial Sequence
  PCR primer 1 for GtTE_607
gcggccgctctagagtgggaggaggaaattgcgtc

SEQ ID NO: 20 - DNA, 35 bp, Artificial Sequence
  PCR primer 1 for GtTE_608
gcggccgctctagaggaggaggaaattgcgtctcg

SEQ ID NO: 21 - DNA, 35 bp, Artificial Sequence
  PCR primer 1 for GtTE_609
gcggccgctctagaggaggaaattgcgtctcgtcc

SEQ ID NO: 22 - DNA, 35 bp, Artificial Sequence
  PCR primer 1 for GtTE_610
gcggccgctctagaggaaattgcgtctcgtcctgg

SEQ ID NO: 23 - DNA, 36 bp, Artificial Sequence
  PCR primer 1 for GtTE_611
gcggccgctctagagattgcgtctcgtcctggactg

SEQ ID NO: 24 - DNA, 375 bp, Artificial Sequence
  Zeocin resistance gene
atggccaagctgaccagcgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactaa

SEQ ID NO: 25 - DNA, 500 bp, Artificial Sequence
  Tubulin promoter
actgcgcatggattgaccgacggccggttgccaacttttggggtcggccccccttttctagcccttgcccgtccagcaattaaaaattatcaacggcataccggcactggaagcttcgggtttacaattttggcttgccttcctaatactgtaccgcggagaacgtatgatattacagaaaaatgccttgcacagttagcgcaaagggaaaacgtttctccgccattgtactttttggaagagggaaagcgattgtaaaatatggctctccgctacgagagtttgggctgttgatacatgtgaaaataagtgtggacgactttgaatgacttgatcaggctgtttgcacatataaccagcgcgcatgcacttctgacatgtcaatgacgaaatttcacacttcaccaataaattgtatccttacgttttgtctttctcacacgcacatatatgatcatagataaaagccaatatcaagaagctgtcttttttgtgaagca

SEQ ID NO: 26 - DNA, 37 bp, Artificial Sequence
  PCR primer No. 26
cttttttgtgaagcaatggccaagttgaccagtgccg

SEQ ID NO: 27 - DNA, 36 bp, Artificial Sequence
  PCR primer No. 27
tttcccccatcccgattagtcctgctcctcggccac

SEQ ID NO: 28 - DNA, 35 bp, Artificial Sequence
  PCR primer No. 28
cgagctcggtacccgactgcgcatggattgaccga

SEQ ID NO: 29 - DNA, 28 bp, Artificial Sequence
  PCR primer No. 29
tgcttcacaaaaaagacagcttcttgat

SEQ ID NO: 30 - DNA, 25 bp, Artificial Sequence
  PCR primer No. 30
tcgggatgggggaaaaaaacctctg

SEQ ID NO: 31 - DNA, 36 bp, Artificial Sequence
  PCR primer No. 31
actctagaggatcccctttcgtaaataaatcagctc

SEQ ID NO: 32 - DNA, 1000 bp, Nannochloropsis oculata
tcgggatgggggaaaaaaacctctgtgtgggctgtcagttgatactattagaggtcttttgttttgtttgtggctgcgtgtgtgtgtttgcatgagaaatagacttgagaatatcggaaggaactttgacatggtaaacgaggaaaagaaaatcttcaaaaaggaataatgggtaaaaacaaggagcaccgggtctctttagaaatgcttctcggcggaaaaccagaaaaaaaggtagaatatgtcgactttttcgcttatcattatagaatgaaagatcgaatggccaagggatttataaattctttctttatgttgtcgtagaacttactttccatcccgagggaggtgtatgcaggccaaaccctctgacatgggcgcaatatctctatgaaaggttgttggaatacattgtccgacctccttcgaggcggagccgcatagttgaagtataggtgcttgcttcatccatctcatgacgctttgccagtgactcactcatgcatgtgacacatttagttctgctcgctcaagcctggcccctcctgacatgcacacattgcacttgtaggtgggccacgtttagtatagacgccacccctgtcgcaccatcggtcccagagcaggagcacgcttccctactcctgtacgctccccctgcttccccccctgctcgtcaacgatggcgacgccagcggctgcgaattacagtgacggcgcggccgctcaggatgacagctcctctccttcaacatctcccaatcttccacccccgcccatgtcgtcgttcgtacggcctatgctgaccgatatgtaccaaattacaatggtcttcgcgtactggaagcaaaagcggcaccaggacagggccatctttgagctctttttccggaagacaccctttaagggagagtttgccattatggccggcattgacgaagtactcaagtacttggcccactttcgcttctccgaggaggagctgatttatttacgaaag

SEQ ID NO: 33 - DNA, 20 bp, Artificial Sequence
  PCR primer No. 33
gggatcctctagagtcgacc

SEQ ID NO: 34 - DNA, 20 bp, Artificial Sequence
  PCR primer No. 34
cgggtaccgagctcgaattc

SEQ ID NO: 35 - DNA, 35 bp, Artificial Sequence
  PCR primer No. 35
cgcggtgttgcgcgcccgctgtggtacttttgtcc

SEQ ID NO: 36 - DNA, 35 bp, Artificial Sequence
  PCR primer No. 36
cgcggtgttgcgcgcattggcggtgcagcattggc

SEQ ID NO: 37 - DNA, 35 bp, Artificial Sequence
  PCR primer No. 37
cgcggtgttgcgcgcccgaaaatcggcaccatcat

SEQ ID NO: 38 - DNA, 35 bp, Artificial Sequence
  PCR primer No. 38
cgcggtgttgcgcgcttctttccgcccaccctgaa

SEQ ID NO: 39 - DNA, 35 bp, Artificial Sequence
  PCR primer No. 39
cgcggtgttgcgcgctgggaggaggaaattgcgtc

SEQ ID NO: 40 - DNA, 35 bp, Artificial Sequence
  PCR primer No. 40
ctcttccacagaagcttattcttttttccgcggaa

SEQ ID NO: 41 - DNA, 35 bp, Artificial Sequence
  PCR primer No. 41
cgagctcggtacccgttcttccgcttgttgctgcc

SEQ ID NO: 42 - DNA, 25 bp, Artificial Sequence
  PCR primer No. 42
tgttgatgcgggctgagattggtgg

SEQ ID NO: 43 - DNA, 36 bp, Artificial Sequence
  PCR primer No. 43
cagcccgcatcaacaatgaagaccgccgctctcctc

SEQ ID NO: 44 - DNA, 28 bp, Artificial Sequence
  PCR primer No. 44
gcgcgcaacaccgcgggtgcgggagaac

SEQ ID NO: 45 - DNA, 21 bp, Artificial Sequence
  PCR primer No. 45
gcttctgtggaagagccagtg

SEQ ID NO: 46 - DNA, 28 bp, Artificial Sequence
  PCR primer No. 46
ggcaagaaaagctgggggaaaagacagg

SEQ ID NO: 47 - DNA, 800 bp, Nannochloropsis oculata
ttcttccgcttgttgctgccgatggcggccatggtctctaagatggagtggatggaggaggaggcgagcgtagcagcaagcgtgagttatacagccaggcacatgtcgcaatccttcggtctcgggcttaaaatccacgcactaatcacgctgggccatgcaaagagcaatgccgaggcccaccacacaaaacgctgtgtcgcgcgttgcggcctgaagcttcatacttcttagtcgccgccaaaagggctcgagagacgagacccgttggcatgaccgatgttgttcgacgcggtttgcttcgtcacagtcgacgtgattcaggaatctggagcctgcagatcatttttttcagcctgatatcgttcttttccactgagaaccatcagaccaccttttcttccattgtgtgaaggagtaggagttgccgtgctgctttgtgggagacatctgcgatggtgaccagcctcccgtcgtctggtcgacgtgacgagcctcttcactgttcttcgacggagagacgcaagcgagacggctctagaccttttggacacgcattctgtgtgtgaactagtggacagtgataccacgtctgaaagctcaccactgcccatggtgcagctacttgtcacaaagttttgactccgtcggtatcaccattcgcgctcgtgtgcctggttgttccgccacgccggcctgccccggggcggggcaatattctaaaatctcacgcaaaacaccgcacttacccctcacacatattcgtgatagaccaccaccaatctcagcccgcatcaaca

SEQ ID NO: 48 - DNA, 99 bp, Nannochloropsis oculata
atgaagaccgccgctctcctcactgtctccaccctcatgggcgcccaggcctttatggcccccgcccccaagttctcccgcacccgcggtgttgcgcgc

SEQ ID NO: 49 - DNA, 613 bp, Nannochloropsis oculata
gcttctgtggaagagccagtggtagtagcagtagcagcagcagtagcagccgcagcactcagtgttggcgcgagagattgtccatcccttcttaacctaccggaagagaaataaggcctttctcccgtagctgtcttcgtttgtttgtgctgattgcttgatatgagagtgttgaattcctgcatcatgtttttctctgtagtcctttcctacccccgtcattttcttttctccctggttcttcttttgtcacccttattttacataaaattttctttgtttatagtgagaggaaggtagagaggggaaaacaagaacaacgaacgcaagcgtgtgaaaggagggcgagtagaagagaaacagatctgttgagcattgagagtggagccgggggaaaggcttgtgtgttgtctttgaaaaagttgtttaaatcacgaatccgttagttctcatgtgtacctctttcactacatgtgatggagaaaacaaaagtgtgaggattaattgaagaaaaagaagagttcgacacgtcaaaccgcccaaaagacgtcacaaagagaacttgattctctttgccgtgttgatcctgtcttttcccccagcttttcttgcc

SEQ ID NO: 50 - DNA, 35 bp, Artificial Sequence
  PCR primer No. 50
ccagcttttcttgccactgcgcatggattgaccga

(19) United States
(12) Patent Application Publication
(10) Pub. No.: US 2017/0335353 A1
(43) Pub. Date: Nov. 23, 2017

(54) Method of Producing Lipid Using Acyl-ACP Thioesterase

(71) Applicant: Kao Corporation, Chuoku, Tokyo (JP)

(72) Inventor: Tatsuro OZAKI, Wakayama-shi, Wakayama (JP)

(73) Assignee: Kao Corporation, Chuoku, Tokyo (JP)

(21) Appl. No.: 15/520,146

(22) PCT Filed: Nov. 6, 2015
(86) PCT No.: PCT/JP2015/081357

§ 371 Date: Apr. 19, 2017

(30) Foreign Application Priority Data
Dec. 5, 2014 (JP) 2014-246573

(51) Int. Cl. C12P 7/64; C12N 9/16

(52) U.S. Cl. CPC C12P 7/64; C12N 9/16; C12Y 301/02

(57) ABSTRACT

[Problems] To provide a method of producing a lipid, containing enhancing productivity of medium chain fatty acids or the lipid containing these medium chain fatty acids as components.

[Means to solve] A method of producing a lipid, containing the steps of:
culturing a transformant in which a gene encoding any one of the following proteins (A) to (C) is introduced into a host, and
collecting a lipid from the cultured product:
(A) a protein consisting of the amino acid sequence of the 611th to 772nd positions set forth in SEQ ID NO: 1;
(B) a protein consisting of an amino acid sequence having 80% or more identity with the amino acid sequence of the 611th to 772nd positions set forth in SEQ ID NO: 1, and having acyl-ACP thioesterase activity; and
(C) a protein containing the amino acid sequence of the protein (A) or (B), and having acyl-ACP thioesterase activity.

[Selected Drawing] None

### TECHNICAL FIELD

The present invention relates to a method of producing a lipid using an acyl-ACP thioesterase. Further, the present invention also relates to an acyl-ACP thioesterase; a gene encoding the same, and a transformant obtained by introducing the gene, for use in this method.

### BACKGROUND ART

Fatty acids are one of the principal components of lipids. In vivo, fatty acids are bonded to glycerin via an ester bond to form lipids such as triacylglycerol. Further, many animals and plants also store and utilize fatty acids as an energy source. These fatty acids and lipids stored in animals and plants are widely utilized for food or industrial use.

For example, higher alcohol derivatives that are obtained by reducing higher fatty acids having approximately 12 to 18 carbon atoms are used as surfactants. Alkyl sulfuric acid ester salts, alkylbenzenesulfonic acid salts and the like are utilized as anionic surfactants. Further, polyoxyalkylene alkyl ethers, alkyl polyglycosides and the like are utilized as nonionic surfactants. These surfactants are used for detergents or disinfectants. Other higher alcohol derivatives, such as alkylamine salts and mono- or dialkyl-quaternary amine salts are commonly used for fiber treatment agents, hair conditioning agents or disinfectants. Further, benzalkonium type quaternary ammonium salts are commonly used for disinfectants or antiseptics. Furthermore, higher alcohols having approximately 18 carbon atoms are also useful as a growth promoter for a plant.

Fatty acids and lipids are widely used for various applications shown above, and therefore, it has been attempted to enhance the productivity of fatty acids or lipids in vivo by using plants and the like. Furthermore, the applications and usefulness of fatty adds depend on the number of carbon atoms. Therefore, controlling of the number of carbon atoms of the fatty acids, namely, a chain length thereof has also been attempted.

For example, a method of accumulating fatty acids having 12 carbon atoms by introducing an acyl-ACP thioesterase derived from Umbellularia californica (California bay) (Patent Literature 1, and Non-Patent Literature 1) has been proposed.

Recently, algae attract attention due to its usefulness in biofuel production. The algae can produce lipids that can be used as the biodiesel fuels through photosynthesis, and do not compete with foods. Therefore, the algae attract attention as next-generation biomass resources. Moreover, the algae are also reported to the effect that the algae have higher lipid productivity and accumulation ability in comparison with plants.

Research has started on a lipid synthesis mechanism of the algae and lipid production technologies utilizing the mechanism, but unclear parts remain in many respects. For example, almost no report has been made so far on the above-mentioned acyl-ACP thioesterase derived from algae, either, and only limited examples of reports are made on genus Nannochloropsis or the like (for example, Patent Literature 2).

### CITATION LIST

### Patent Literatures

Patent Literature 1: WO 92/20236

Patent Literature 2: WO 2014/103930

### Non-Patent Literatures

Non-Patent literature 1; Voelker T A, et al., Science, 1992, vol 257 (5066), p. 72-74.

### SUMMARY OF INVENTION

The present invention relates to a method of producing a lipid, containing the steps of:

culturing a transformant in which a gene encoding any one of the following proteins (A) to (C) is introduced into a host, and

collecting a lipid from the cultured product:

(A) a protein consisting of the amino add sequence of the 611th to 722nd positions set forth in SEQ ID NO: 1;
(B) a protein consisting of an amino acid sequence having 80% or more identity with the amino acid sequence of the 811th to 722nd positions set forth in SEQ ID NO: 1, and having acyl-ACP thioesterase activity; and
(C) a protein containing the protein of the amino acid sequence of the protein (A) or (B), and having acyl-ACP thioesterase activity.

The present invention relates to the proteins (A) to (C) (hereinafter, also referred to as “the protein of the present invention” or “the acyl-ACP thioesterase of the present invention”).

Further, the present invention relates to a gene encoding any one of the proteins (A) to (C) (hereinafter, also referred to as “the gene of the present invention”).

Furthermore, the present invention relates to a transformant, which is obtained by introducing a gene encoding any one of the proteins (A) to (C) into a host.

Other and further features and advantages of the invention will appear more fully from the following description.

### MODE FOR CARRYING OUT THE INVENTION

The present invention is contemplated for providing a method of producing a lipid, containing enhancing productivity of medium chain fatty acids or the lipid containing these fatty acids as components.

Further, the present invention is contemplated for providing a novel acyl-ACP thioesterase derived from algae and a gene encoding this, which can be suitably used for the method.

Furthermore, the present invention is contemplated for providing a transformant in which the expression of the gene is promoted and productivity of a lipid or fatty acid composition is changed.

The present inventor conducted research on novel acyl-ACP thioesterases derived from algae. As a result, the present inventor found a novel acyl-ACP thioesterase and an acyl-ACP thioesterase gene encoding this from a cryptophyte. Further, as a result of conducting transformation by using the acyl-ACP thioesterase gene, the present inventor found that, in transformants, the ratio of the content of specific fatty acids to total fatty acid components in the lipid is significantly improved.

The present invention was completed based on these findings.

The present invention can provide a novel acyl-ACP thioesterase, a gene encoding this, and a transformant in which the gene is introduced. A method of producing a lipid using the transformant according to the present invention is excellent in productivity of medium chain fatty acids or the lipid containing these fatty acids as components. In particular, a method of producing a lipid according to the present invention is excellent in productivity of the fatty acids having 8 to 16 carbon atoms, preferably 8 to 14 carbon atoms, more preferably 10 to 14 carbon atoms, further preferably 12 to 14 carbon atoms, furthermore preferably 12 or 14 carbon atoms, and furthermore preferably 12 carbon atoms, and the lipid containing these fatty acids as components.

The acyl-ACP thioesterase, the gene encoding this acyl-ACP thioesterase, the transformant and the method of producing a lipid of the present invention can be suitably used for the industrial production of fatty acids or lipids.

In the present invention, the term “lipid(s)” covers simple lipids, complex lipids and derived lipids. Specifically, “lipid(s)” covers fatly acids, aliphatic alcohols, hydrocarbons (such as alkanes), neutral lipids (such as triacylglycerol), wax, ceramides, phospholipids, glycolipids, sulfolipids and the like.

In the present specification, the description of “Cxy” for the fatty acid or the acyl group constituting the fatty acid means that the number of carbon atoms is “x” and the number of double bonds is “y”. The description of “Cx” means a fatty acid or an acyl group having “x” as the number of carbon atoms.

In the present specification, the identify of the nucleotide sequence and the amino acid sequence is calculated through the Lipman-Pearson method (Science, 1985, vol. 227, p. 1435-1441). Specifically, the identity can be determined through use of a homology analysis (search homology) program of genetic information processing software Genetyx-Win with Unit size to compare (ktup) being set to 2.

It should be note that, in this description, the “stringent conditions” includes, for example, the method described in Molecular Cloning—A LABORATORY MANUAL THIRD EDITION [Joseph Sambrook, David W. Russell, Cold Spring Harbor Laboratory Press], and examples thereof include conditions where hybridization is performed by incubating a solution containing 6×SSC (composition of 1×SSC: 0.15M sodium chloride, 0.015M sodium citrate, pH 7.0), 0.5% SDS, 5× Denhardt and 100 mg/mL herring sperm DNA together with a probe at 65° C. for 8 to 18 hours.

In the present specification, any numerical expressions in a style of “ . . . to . . . ” will be used to indicate a range including the lower and upper limits represented by the numerals given before and after “to”, respectively.

Furthermore, in the present specification, the term “medium chain” means that the number of carbon atoms of the fatty acid or the fatty acid residue is 8 or more and 16 or less.

Hereinafter, the acyl-ACP thioesterase, the transformant using the same, and the method of producing a lipid of the present invention are described below in order.

### 1. Acyl-ACP Thioesterase

The protein of the present invention includes a protein having at least amino acid sequence of the 611th to 772nd positions in the amino acid sequence set forth in SEQ ID NO: 1, and a protein functionally equivalent to the protein.

The acyl-ACP (acyl carrier protein) thioesterase is an enzyme involved in the biosynthesis pathway of fatty acids and derivatives thereof (such as triacylglycerol (triglyceride)). This enzyme hydrolyzes a thioester bond of an acyl-ACP to form a free fatty acid in a plastid such as a chloroplast of plant and alga or in a cytoplasm of bacteria, fungus and animal. The acyl-ACP is a composite composed of an acyl group (fatty acid residue) and an acyl carrier protein, and is an intermediate in the process of fatty acid biosynthesis. The function of the acyl-ACP thioesterase terminates the synthesis of the fatty acid on the ACP, and then the thus-produced fatty acids are supplied to the synthesis of triacylglycerol and the like.

To date, several acyl-ACP thioesterases having different reaction specificities depending on the number of carbon atoms and the number of unsaturated bonds of the acyl group (fatty acid residue) of the acyl-ACP substrate are identified. Therefore, acyl-ACP thiosterase is considered to be an important factor in determining the fatty acid composition in vivo.

The “acyl-ACP thioesterase activity” in the present invention means an activity of hydrolyzing the thioester bond of the acyl-ACP.

Specific examples of the protein of the present invention include the following proteins (A) to (C).

(A) A protein consisting of the amino acid sequence of the 811th to 772nd positions set forth in SEQ ID NO: 1.
(B) A protein consisting of an amino acid sequence having 80% or more identity with the amino acid sequence of the 811th to 772nd positions set forth in SEQ ID NO: 1, and having acyl-ACP thioesterase activity.
(C) A protein containing the amino acid sequence of the protein (A) or (B), and having acyl-ACP thioesterase activity.

The amino acid sequence set forth in SEQ ID NO: 1 is an amino acid sequence of the acyl-ACP thioesterase (hereinafter, also abbreviated as “GtTE”) derived from Guillardia theta, which is a kind of cryptophyte.

Although the genome sequence information of the Guillardia theta is open, the function of the amino acid sequence set forth in SEQ ID NO: 1 has not been identified so far. The present inventor has identified the protein consisting of the amino acid sequence set forth in SEQ ID NO: 1 as an acyl-ACP thioesterase. Moreover, the present inventor has compared the amino acid sequence set forth in SEQ ID NO: 1 with that of other well-known acyl-ACP thioesterases, and has found that the sequence identity (homology) thereof is extremely low.

Furthermore, the present inventor found that the region of the 611th to 772nd positions in the amino acid sequence set forth in SEQ ID NO: 1 is an important for acting the acyl-ACP thioesterase, and sufficient region for exhibiting the acyl-ACP thioesterase activity. That is, the protein consisting of the amino acid sequence of the 811th to 772nd positions set forth in SEQ ID NO: 1 and a protein containing the amino acid sequence have the acyl-ACP thioesterase activity.

The protein (A) consists of a region sufficient for this acyl-ACP thioesterase activity, and acts as the acyl-ACP thioesterase.

The protein (B) consists of an amino acid sequence having 80% or more identity with the amino acid sequence of the 611th to 772nd positions set forth in SEQ ID NO: 1, and has acyl-ACP thioesterase activity.

In general, it is known that an amino acid sequence encoding an enzyme protein does not necessarily exhibit enzyme activity unless the sequence in the whole region is conserved, and there exists a region in which the enzyme activity is not influenced even if the amino acid sequence is changed. In such a region which is not essential to the enzyme activity, even if the mutation of the amino acid, such as deletion, substitution, insertion and addition thereof is introduced thereinto, the activity inherent to the enzyme can be maintained. Also in the present invention, such a protein can be used in which the acyl-ACP thioesterase activity is kept and a part of the amino acid sequence is subjected to mutation.

From a viewpoint of acyl-ACP thioesterase activity, the protein (B) has preferably 85% or more identity, more preferably 90% or more identity, further preferably 95% or more identity, furthermore preferably 96% or more identity, furthermore preferably 97% or more identity, furthermore preferably 98% or more identity, and furthermore preferably 99% or more identity, with the amino acid sequence of the 611th to 772nd positions set forth in SEQ: ID NO: 1.

Further, with respect to the protein (B), specific examples of the amino acid sequence having 80% or more identity with the amino acid sequence of the 611th to 772nd positions set forth in SEQ ID NO: 1 include an amino acid sequence in which 1 or several amino acids, preferably 1 or more and 20 or less amino acids, more preferably 1 or more and 15 or less amino acids, further preferably 1 or more and 10 or less amino acids, furthermore preferably 1 or more and 8 or less amino acids, furthermore preferably 1 or more and 5 or less amino acids, furthermore preferably 1 or more and 4 or less amino acids, furthermore preferably 1 or more and 3 or less amino acids, and furthermore preferably 1 or 2 amino acids, are deleted, substituted, inserted or added in the amino acid sequence of the 611th to 772nd positions set forth in SEQ ID NO: 1.

A method of introducing the mutation such as deletion, substitution, insertion or addition into an amino acid sequence includes a method of, for example, introducing a mutation into a nucleotide sequence encoding the amino acid sequence. The method of introducing a mutation into a nucleotide sequence is described later.

The protein (C) contains the amino acid sequence of the protein (A) or (B) as a part of the amino acid sequence of the protein (C), and exhibits acyl-ACP thioesterase activity. The protein (C) may include a sequence other than the amino acid sequence of the protein (A) or (B).

Specific examples of the sequence other than the amino acid sequence of the protein (A) or (B) in the amino acid sequence that constitutes the protein (C) include an arbitrary amino acid sequence other than the 611th to 772nd positions set forth in SEQ ID NO: 1, an amino acid sequence having 80% or more identity, preferably 85% or more identity, more preferably 90% or more identity, further preferably 95% or more identity, furthermore preferably 96% or more identity, furthermore preferably 97% or more identity, furthermore preferably 98% or more identity, and furthermore preferably 99% or more identify, with the arbitrary amino acid sequence other than the 611th to 772nd positions set forth in SEQ ID NO: 1, or an amino acid sequence in which one or several amino acids, preferably 1 or more and 20 or less amino acids, more preferably 1 or more and 15 or less amino acids, further preferably 1 or more and 10 or less amino acids, furthermore preferably 1 or more and 8 or less amino acids, furthermore preferably 1 or more and 5 or less amino acids, furthermore preferably 1 or more and 4 or less amino acids, furthermore preferably 1 or more and 3 or less amino acids, and furthermore preferably 1 or 2 amino acids, are deleted, substituted, inserted or added into these sequences. These sequences are preferably added to the N-terminal side of the amino acid sequence of the protein (A) or (B).

Moreover, the protein (C) also preferably includes a protein consisting of an amino acid sequence formed such that a signal peptide engaging in transport or secretion of the protein is added to the amino acid sequence of the protein (A) or (B). Specific examples of addition of the signal peptide include addition to an N-terminal of chloroplast transit signal peptide.

The protein (C) may be a protein consisting of an amino acid sequence in which amino acids on an N-terminal side are deleted at an arbitrary position of the 1st to 610th positions set forth in SEQ ID NO: 1.

Further, from a viewpoint of the productivity of specific fatty acids, for example medium chain fatty adds, the protein (C) is preferably the following proteins (C1) to (C20).

(C1) A protein consisting of the amino acid sequence of the 1st to 772nd positions set forth in SEQ ID NO: 1.
(C2) A protein consisting of the amino acid sequence of the 487th to 772nd positions set forth in SEQ ID NO: 1.
(C3) A protein consisting of the amino acid sequence of the 488th to 772nd positions set forth in SEQ ID NO: 1.
(C4) A protein consisting of the amino acid sequence of the 497th to 772nd positions set forth in SEQ ID NO: 1.
(C5) A protein consisting of the amino acid sequence of the 507th to 772nd positions set forth in SEQ ID NO: 1.
(C6) A protein consisting of the amino acid sequence of the 517th to 772nd positions set forth in SEQ ID NO: 1.
(C7) A protein consisting of the amino acid sequence of the 527th to 772nd positions set forth in SEQ: ID NO: 1.
(C8) A protein consisting of the amino acid sequence of the 537th to 772nd positions set forth in SEQ ID NO: 1.
(C9) A protein consisting of the amino acid sequence of the 547th to 772nd positions set forth in SEQ ID NO: 1.
(C10) A protein consisting of the amino acid sequence of the 557th to 772nd positions set forth in SEQ ID NO: 1.
(C11) A protein consisting of the amino acid sequence of the 587th to 772nd positions set forth in SEQ ID NO: 1.
(C12) A protein consisting of the amino acid sequence of the 577th to 772nd positions set forth in SEQ ID NO: 1.
(C13) A protein consisting of the amino add sequence of the 587th to 772nd positions set forth in SEQ ID NO: 1.
(C14) A protein consisting of the amino acid sequence of the 597th to 772nd positions set forth in SEQ ID NO: 1.
(C15) A protein consisting of the amino acid sequence of the 807th to 772nd positions set forth in SEQ ID NO: 1.
(C16) A protein consisting of the amino acid sequence of the 608th to 772nd positions set forth in SEQ ID NO: 1.
(C17) A protein consisting of the amino acid sequence of the 609th to 772nd positions set forth in SEQ ID NO: 1.
(C18) A protein consisting of the amino acid sequence of the 610th to 772nd positions set forth in SEQ ID NO: 1.
(C19) A protein consisting of an amino acid sequence having 80% or more identity, preferably 85% or more identity, more preferably 90% or more identity, further preferably 95% or more identity, furthermore preferably 96% or more identity, furthermore preferably 97% or more identity, furthermore preferably 98% or more identity, and furthermore preferably 99% or more identity, with the amino acid sequence of any one of the proteins (C1) to (C18), and having acyl-ACP thioesterase activity.
(C20) A protein consisting of an amino acid sequence in which 1 or several amino acids, preferably 1 or more and 20 or less amino acids, more preferably 1 or more and 15 or less amino acids, further preferably 1 or more and 10 or less amino acids, furthermore preferably 1 or more and 8 or less amino acids, furthermore preferably 1 or more and 5 or less amino acids, furthermore preferably 1 or more and 4 or less amino acids, furthermore preferably 1 or more and 3 or less amino acids, and furthermore preferably 1 or 2 amino acids, are deleted, substituted, inserted or added to the amino acid sequence of any one of the proteins (C1) to (C18), and having acyl-ACP thioesterase activity.

The present inventor confirmed that the proteins (C1) to (C18) have the acyl-ACP thioesterase activity.

The protein (C) of the present invention is preferably the protein (C13), the protein (C14), the protein (C15), the protein (C16), the protein (C17), a protein consisting of the amino acid sequence having 80% or more identity, preferably 85% or more identity, more preferably 90% or more identify, further preferably 95% or more identity, furthermore preferably 96% or more identity, furthermore preferably 97% or more identity, furthermore preferably 98% or more identity, and furthermore preferably 99% or more identity, with the amino acid sequence of any one of the proteins (C13) to (C17), and having acyl-ACP thioesterase activity, or a protein consisting of the amino acid sequence in which 1 or several amino acids, preferably 1 or more and 20 or less amino acids, more preferably 1 or more and 15 or less amino acids, further preferably 1 or more and 10 or less amino acids, furthermore preferably 1 or more and 8 or less amino acids, furthermore preferably 1 or more and 5 or less amino acids, furthermore preferably 1 or more and 4 or less amino acids, furthermore preferably 1 or more and 3 or less amino acids, and furthermore preferably 1 or 2 amino acids, are deleted, substituted, inserted or added to the amino acid sequence of any one of the proteins (C13) to (C17), and having acyl-ACP thioesterase activity.

The acyl-ACP thioesterase activity of the protein can be confirmed by, for example, introducing a DNA produced by linking the acyl-ACP thioesterase gene to the downstream of a promoter which functions in a host cell such as Escherichia coli, into a host cell which lacks a fatty acid degradation system, culturing the thus-obtained cell under the conditions suitable for the expression of the introduced acyl-ACP thioesterase gene, and analyzing any change caused thereby in the fatty acid composition of the host cell or the cultured liquid by using a gas chromatographic analysis or the like.

Alternatively, the acyl-ACP thioesterase activity can be measured by introducing a DNA produced by linking the acyl-ACP thioesterase gene to the downstream of a promoter which functions in a host cell such as Escherichia coli, into a host cell, culturing the thus-obtained cell under the conditions suitable for the expression of the introduced acyl-ACP thioesterase gene, and subjecting a disruption liquid of the cell to a reaction which uses acyl-ACPs, as substrates, prepared according to the method of Yuan et at. (Yuan L. et al., Proc. Natl. Acad. Sci. U.S.A., 1995, vol. 92 (23), p. 10639-10643).

There are no particular limitations on the method for obtaining the protein of the present invention, and the protein can be obtained by chemical techniques, genetic engineering techniques or the like that are ordinarily carried out. For example, a natural product-derived protein can be obtained through isolation, purification and the like from Guillardia theta. Furthermore, protein synthesis may be carried out by chemical synthesis, or a recombinant protein may also be produced by gene recombination technologies. In the case of producing a recombinant protein, the acyl-ACP thioesterase gene described below can be used.

Moreover, the cryptophyte such as Guillardia theta can also be obtained from culture collection such as private or public research institutes. For example, the cryptophyte can be obtained from National Center for Marine Algae and Microbiota (NCMA, previous name: CCMP). The culture collection of algae at University of Texas at Austin (UTEX), National Institute for Environmental Studies (NIES), Culture Collection of Algae and Protozoa (CCAP), or Australian National Algae Culture Collection (CSIRO).

### 2. Acyl-ACP Thioesterase Gene

The acyl-ACP thioesterase gene of the present invention is a gene encoding any one of the proteins (A) to (C).

Examples of the gene encoding any one of the proteins (A) to (C) include a gene consisting of the nucleotide sequence set forth in SEQ ID NO: 2 or 3.

The nucleotide sequence set forth in SEQ ID NO: 2 is an example of the nucleotide sequence of the gene encoding the wild type acyl-ACP thioesterase derived from Guillardia theta. The nucleotide sequence of the 1,831st to 2,316th positions set forth in SEQ ID NO: 2 encodes the amino acid sequence of the 611th to 772nd positions set forth in SEQ ID NO: 1. In addition, a nucleotide sequence of the 2,317th to 2,319th positions set forth in SEQ ID NO: 2 is a termination codon, which does not correspond to any amino acids.

The nucleotide sequence set forth in SEQ ID NO: 3 is a nucleotide sequence subjected to codon optimization along with the using frequency of the codon of Escherichia coli based on the amino acid sequence set forth in SEQ ID NO: 1. The nucleotide sequence of the 1st to 858th positions set forth in SEQ ID NO: 3 encodes the amino acid sequence of the 487th to 772nd positions set forth in SEQ ID NO: 1. The nucleotide sequence of the 373rd to 858th positions set forth in SEQ ID NO: 3 encodes the amino acid sequence of the 611th to 772nd positions set forth in SEQ ID NO: 1. In addition, a nucleotide sequence of the 859th to 861st positions set forth in SEQ ID NO: 3 is a termination codon, which does not correspond to any amino acids.

Specific examples of the gene encoding any one of the proteins (A) to (C) include a gene consisting of any one of the following DNAs (a) to (f). However, the present invention is not limited thereto.

(a) A DNA consisting of the nucleotide sequence of the 1,831st to 2,319th positions set forth in SEQ ID NO: 2.
(b) A DNA consisting of a nucleotide sequence having 80% or more identity with the nucleotide sequence of the 1,831st to 2,319th positions set forth in SEQ ID NO: 2, and encoding a protein having acyl-ACP thioesterase activity.
(c) A DNA containing the nucleotide sequence of the DNA (a) or (b), and encoding a protein having acyl-ACP thioesterase activity.
(d) A DNA consisting of the nucleotide sequence of the 373rd to 861st positions set forth in SEQ ID NO: 3.
(e) A DNA consisting of a nucleotide sequence having 80% or more identity with the nucleotide sequence of the 373rd to 861st positions set forth in SEQ ID NO: 3, and encoding a protein having acyl-ACP thioesterase activity.
(f) A DNA containing the nucleotide sequence of the DNA (d) or (e), and encoding a protein having acyl-ACP thioesterase activity.

From a viewpoint of acyl-ACP thioesterase activity, the DNA (b) has preferably 85% or more identity, more preferably 90% or more identity, further preferably 95% or more identity, furthermore preferably 96% or more identity, furthermore preferably 97% or more identity, furthermore preferably 98% or more identity, and furthermore preferably 99% or more identity, with the nucleotide sequence of the 1,831st to 2,319th positions set forth in SEQ ID NO: 2.

Further, with respect to the DNA (b), specific examples of the nucleotide sequence having 80% or more identity with the nucleotide sequence of the 1,831st to 2,319th positions set forth in SEQ ID NO: 2 include a nucleotide sequence in which 1 or several nucleotides, preferably 1 or more and 20 or less nucleotides, more preferably 1 or more and 15 or less nucleotides, further preferably 1 or more and 10 or less nucleotides, furthermore preferably 1 or more and 8 or less nucleotides, furthermore preferably 1 or more and 5 or less nucleotides:, furthermore preferably 1 or more and 4 or less nucleotides, furthermore preferably 1 or more and 3 or less nucleotides, and furthermore preferably 1 or 2 nucleotides, are deleted, substituted, inserted or added in the nucleotide sequence of the 1,831st to 2,319th positions set forth in SEQ ID NO: 2.

From a viewpoint of acyl-ACP thioesterase activity, the DNA (e) has preferably 85% or more identity, more preferably 90% or more identity, further preferably 95% or more identity, furthermore preferably 96% or more identity, furthermore preferably 97% or more identity, furthermore preferably 98% or more identity, and furthermore preferably 99% or more identity, with the nucleotide sequence of the 373rd to 861st positions set forth in SEQ ID NO: 3.

Further, with respect to the DNA (e), specific examples of the nucleotide sequence having 80% or more identity with the nucleotide sequence of the 373rd to 861st positions set forth in SEQ ID NO: 3 include a nucleotide sequence in which 1 or several nucleotides, preferably 1 or more and 20 or less nucleotides, more preferably 1 or more and 15 or less nucleotides, further preferably 1 or more and 10 or less nucleotides, furthermore preferably 1 or more and 8 or less nucleotides, furthermore preferably 1 or more and 5 or less nucleotides, furthermore preferably 1 or more and 4 or less nucleotides, furthermore preferably 1 or more and 3 or less nucleotides, and furthermore preferably 1 or 2nucleotides, are deleted, substituted, inserted or added in the nucleotide sequence of the 373rd to 861st positions set forth in SEQ ID NO: 3.

A method of introducing the mutation such as deletion, substitution, insertion or addition info a nucleotide sequence includes a method of introducing a site-specific mutation, for example. Specific examples of the method of introducing the site-specific mutation include a method of utilizing the Splicing overlap extension (SOE) PCR (Norton et al., Gene, 1989, vol. 77, p. 61-68), the ODA method (Hashimoto-Gotoh et al., Gene, 1995, vol. 152, p. 271-276), and the Kunkel method (Kunkel, T. A., Proc. Natl. Acad. Sci. USA, 1985, vol. 82, p. 488). Further, commercially available kits such as Site-Directed Mutagenesis System Mutan-SuperExpress Km kit (manufactured by Takara Bio), Transformer TM Site-Directed Mutagenesis kit (manufactured by Clonetech Laboratories), and KOD-Plus-Mutagenesis Kit (manufactured by Toyobo) can also be utilized. Furthermore, a gene containing a desired mutation can also be obtained by introducing a genetic mutation at random, and then performing an evaluation of the enzyme activities and a gene analysis thereof by an appropriate method.

Furthermore, the DNA (b) is also preferably a DNA capable of hybridizing with a DNA consisting of a nucleotide sequence complementary with the DNA (a) under a stringent condition, and encoding a protein having acyl-ACP thioesterase activity.

In a similar manner, the DNA (e) is also preferably a DNA capable of hybridizing with a DNA consisting of a nucleotide sequence complementary with the DNA (d) under a stringent condition, and encoding a protein having acyl-ACP thioesterase activity.

The DNA (e) contains the nucleotide sequence of the DNA (a) or (b) as a part of the nucleotide sequence of the DNA (c), and encodes a protein having acyl-ACP thioesterase activity. The DNA (c) may include a sequence other than the nucleotide sequence of the DNA (a) or (b).

Specific examples of the sequence other than the nucleotide sequence of the DNA (a) or (b) in the nucleotide sequence of the DNA (c) include an arbitrary nucleotide sequence other than the 1,831st to 2,319th positions set forth in SEQ ID NO: 2, a nucleotide sequence having 80% or more identity, preferably 85% or more identity, more preferably 90% or more identity, further preferably 95% or more identity, furthermore preferably 96% or more identity, furthermore preferably 97% or more identity, furthermore preferably 98% or more identity, and furthermore preferably 99% or more identity, with the arbitrary nucleotide sequence other than the 1,831st to 2,319th positions set forth in SEQ ID NO: 2, or a nucleotide sequence in which one or several nucleotides, preferably 1 or more and 20 or less nucleotides, more preferably 1 or more and 15 or less nucleotides, further preferably 1 or more and 10 or less nucleotides, furthermore preferably 1 or more and 8 or less nucleotides, furthermore preferably 1 or more and 5 or less nucleotides, furthermore preferably 1 or more and 4 or less nucleotides, furthermore preferably 1 or more and 3 or less nucleotides, and furthermore preferably 1 or 2 nucleotides, are deleted, substituted, inserted or added into an arbitrary nucleotide sequence other than the 1,831st to 2,319th positions set forth in SEQ ID NO. 2.

Moreover, the sequence other than the nucleotide sequence of the DNA (a) or (b) also preferably includes a nucleotide sequence encoding a signal peptide engaging in transport or secretion of the protein. Specific example of the signal peptide includes the proteins described in the protein (C).

These sequences are preferably added to the 5′-terminal side of the nucleotide sequence of the DNA (a) or (b).

The DNA (c) may be a DNA consisting of a nucleotide sequence in which nucleotides on a 5′-terminal side are deleted at an arbitrary position of the 1st to 1,830th positions set forth in SEQ ID NO: 2.

Further, from a viewpoint of the productivity of specific fatty acids, for example medium chain fatty acids, the DNA (c) is preferably the following DNAs (c1) to (c20).

(c1) A DNA consisting of the nucleotide sequence of the 1st to 2,319th positions set forth in SEQ ID NO: 2.
(c2) A DNA consisting of the nucleotide sequence of the 1,459th to 2,319th positions set forth in SEQ ID NO: 2.
(c3) A DNA consisting of the nucleotide sequence of the 1,462nd to 2,319th positions set forth in SEQ ID NO: 2.
(c4) A DNA consisting of the nucleotide sequence-of the 1,489th to 2,319th positions set forth in SEQ: ID NO: 2.
(c5) A DNA consisting of the nucleotide sequence of the 1,519th to 2,319th positions set forth in SEQ ID NO: 2.
(c6) A DNA consisting of the nucleotide sequence of the 1,549th to 2,319th positions set forth in SEQ ID NO: 2.
(c7) A DNA consisting of the nucleotide sequence of the 1,579th to 2,319th positions set forth in SEQ ID NO: 2.
(c8) A DNA consisting of the nucleotide sequence of the 1,609th to 2,319th positions set forth in SEQ ID NO: 2.
(c9) A DNA consisting of the nucleotide sequence of the 1,639th to 2,319th positions set forth in SEQ ID NO: 2.
(c10) A DNA consisting of the nucleotide sequence of the 1,689th to 2,319th positions set forth in SEQ ID NO: 2.
(c11) A DNA consisting of the nucleotide sequence of the 1,699th to 2,319th positions set forth in SEQ ID NO: 2.
(c12) A DNA consisting of the nucleotide sequence of the 1,729th to 2,319th positions set forth in SEQ ID NO: 2.
(c13) A DNA consisting of the nucleotide sequence of the 1,759th to 2,319th positions set forth in SEQ ID NO: 2.
(c14) A DNA consisting of the nucleotide sequence of the 1,789th to 2,319th positions set forth in SEQ ID NO: 2.
(c15) A DNA consisting of the nucleotide sequence of the 1,819th to 2,319th positions set forth in SEQ ID NO: 2.
(c18) A DNA consisting of the nucleotide sequence of the 1,822nd to 2,319th positions set forth in SEQ ID NO: 2.
(c17) A DNA consisting of the nucleotide sequence of the 1,825th to 2,319th positions set forth in SEQ ID NO: 2.
(c18) A DNA consisting of the nucleotide sequence of the 1,828th to 2,319th positions set forth in SEQ ID NO: 2.
(c19) A DNA consisting of a nucleotide sequence having 80% or more identity, preferably 85% or more identity, more preferably 90% or more identity, further preferably 95% or more identity, furthermore preferably 96% or more identity, furthermore preferably 97% or more identity, furthermore preferably 98% or more identity, and furthermore preferably 99% or more identity, with the nucleotide sequence of any one of the DNAs (c1) to (c18), and encoding a protein having acyl-ACP thioesterase activity.
(c20) A DNA consisting of a nucleotide sequence in which 1 or several nucleotides, preferably 1 or more and 20 or less nucleotides, more preferably 1 or more and 15 or less nucleotides, further preferably 1 or more and 10 or less nucleotides, furthermore preferably 1 or more and 8 or less nucleotides, furthermore preferably 1 or more and 5 or less nucleotides, furthermore preferably 1 or more and 4 or less nucleotides, furthermore preferably 1 or more and 3 or less nucleotides, and furthermore preferably 1 or 2 nucleotides, are deleted, substituted, inserted or added to the nucleotide sequence of any one of the DNAs (c1) to (c18), and encoding a protein having acyl-ACP thioesterase activity.

The present inventor confirmed that the gene consisting of any one of the DNAs (c1) to (c18) encodes a protein having acyl-ACP thioesterase activity.

The DNA (f) contains the nucleotide sequence of the DNA (d) or (e) as a part of the nucleotide sequence of the DNA (c), and encodes a protein having acyl-ACP thioesterase activity. The DNA (f) may include a sequence other than the nucleotide sequence of the DNA (d) or (e).

Specific examples of the sequence other than the nucleotide sequence of the DNA (d) or (e) in the nucleotide sequence of the DNA (f) include an arbitrary nucleotide sequence other than the 373rd to 861 st positions set forth in SEQ ID NO: 3, a nucleotide sequence having 80% or more identity, preferably 85% or more identity, more preferably 90% or more identity, further preferably 95% or more identity, furthermore preferably 96% or more identity, furthermore preferably 97% or more identity, furthermore preferably 98% or more identity, and furthermore preferably 99% or more identity, with the arbitrary nucleotide sequence other than the 373rd to 861st positions set forth in SEQ ID NO: 3, or a nucleotide sequence in which one or several nucleotides, preferably 1 or more and 20 or less nucleotides. more preferably 1 or more and 15 or less nucleotides, further preferably 1 or more and 10 or less nucleotides, furthermore preferably 1or more and 8 or less nucleotides, furthermore preferably 1 or more and 5 or less nucleotides, furthermore preferably 1 or mere and 4 or less nucleotides, furthermore preferably 1 or more and 3 or less nucleotides, and furthermore preferably 1 or 2 nucleotides, are deleted, substituted, inserted or added into an arbitrary nucleotide sequence other than the 373rd to 861st positions set forth in SEQ ID NO: 3.

Moreover, the sequence other than the nucleotide sequence of the DNA (d) or (e) also preferably includes a nucleotide sequence encoding a signal peptide engaging in transport or secretion of the protein. Specific example of the signal peptide includes the proteins described in the protein (C).

These sequences are preferably added to the 5′-terminal side of the nucleotide sequence of the DNA (d) or (e).

The DNA (f) may be a DNA consisting of a nucleotide sequence in which nucleotides on a 5′-terminal side are deleted at an arbitrary position of the 1st to 372nd positions set forth in SEQ ID NO: 3.

Further, from a viewpoint of the productivity of specific fatty acids, for example medium chain fatty acids, the DNA (f) is preferably the following DNAs (f1) to (f19).

(f1) A DNA consisting of the nucleotide sequence of the 1st to 861 st positions set forth in SEQ ID NO: 3.
(f2) A DNA consisting of the nucleotide sequence of the 4th to 861st positions set forth in SEQ ID NO: 3.
(f3) A DNA consisting of the nucleotide sequence of the 31st to 861st positions set forth in SEQ ID NO: 3.
(f4) A DNA consisting of the nucleotide sequence of the 61st to 861st positions set forth in SEQ ID NO: 3.
(f5) A DNA consisting of the nucleotide sequence of the 91st to 861st positions set forth in SEQ ID NO: 3.
(f6) A DNA consisting of the nucleotide sequence of the 121st to 861st positions set forth in SEQ ID NO: 3.
(f7) A DNA consisting of the nucleotide sequence of the 151st to 861st positions set forth in SEQ ID NO: 3.
(f8) A DNA consisting of the nucleotide sequence of the 181st to 861st positions set forth in SEQ ID NO: 3.
(f9) A DNA consisting of the nucleotide sequence of the 211th to 861 st positions set forth in SEQ ID NO: 3.
(f10) A DNA consisting of the nucleotide sequence of the 241st to 861st positions set forth in SEQ ID NO: 3.
(f11) A DNA consisting of the nucleotide sequence of the 271st to 861st positions set forth in SEQ ID NO: 3.
(f12) A DNA consisting of the nucleotide sequence of the 301st to 861st positions set forth in SEQ ID NO: 3.
(f13) A DNA consisting of the nucleotide sequence of the 331st to 861st positions set forth in SEQ ID NO: 3.
(f14) A DNA consisting of the nucleotide sequence of the 361st to 861st positions set forth in SEQ ID NO: 3.
(f15) A DNA consisting of the nucleotide sequence of the 364th to 861st positions set forth in SEQ ID NO: 3.
(f16) A DNA consisting of the nucleotide sequence of the 367th to 861 st positions set forth in SEQ ID NO: 3.
(f17 ) A DNA consisting of the nucleotide sequence of the 370th to 861st positions set forth in SEQ ID NO: 3.
(f18) DNA consisting of a nucleotide sequence having 80% or more identity, preferably 85% or more identity, more preferably 90% or more identity, further preferably 95% or more identity, furthermore preferably 96% or more identity, furthermore preferably 97% or more identity, furthermore preferably 98% or more identity, and furthermore preferably 99% or more identity, with the nucleotide sequence of any one of the DNAs (f1) to (f17), and encoding a protein having acyl-ACP thioesterase activity.
(f19) A DNA consisting of a nucleotide sequence in which 1 or several nucleotides, preferably 1 or more and 20 or less nucleotides, more preferably 1 or more and 15 or less nucleotides, further preferably 1 or more and 10 or less nucleotides, furthermore preferably 1 or more and 8 or less nucleotides, furthermore preferably 1 or more and 5 or less nucleotides, furthermore preferably 1 or more and 4 or less nucleotides, furthermore preferably 1 or more and 3 or less nucleotides, and furthermore preferably 1 or 2 nucleotides, are deleted, substituted, inserted or added to the nucleotide sequence of any one of the DNAs (f1) to (f17), and encoding a protein having acyl-ACP thioesterase activity.

The present inventor confirmed that the gene consisting of any one of the DNAs (f1) to (f17) encodes a protein having acyl-ACP thioesterase activity.

There are no particular limitations on the method for obtaining the acyl-ACP thioesterase gene of the present invention, and the gene can fee obtained by genetic engineering techniques that are ordinarily carried out. For example, the gene can be obtained by artificial synthesis based on the amino acid sequence set forth in SEQ ID NO: 1 or the nucleotide sequence set forth in SEQ ID NO: 2 or 3. The artificial synthesis of a gene can be achieved by utilizing, for example, the services of Eurofins Genomics. Furthermore, the gene can also be obtained by cloning from Guillardia theta. The cloning can be carried out by, for example, the methods described in Molecular Cloning—A LABORATORY MANUAL THIRD EDITION [Joseph Sambrook, David W. Russell, Cold Spring Harbor Laboratory Press (2001)].

### 3. Transformant

The transformant of the present invention is a transformant in which the expression of a gene encoding any one of the proteins (A) to (C) is promoted.

In the transformant, the ability to produce lipids, particularly the ability to produce medium chain fatty acids or lipids containing these medium chain fatty acids as components (productivity of medium chain fatty acids or lipids containing these medium chain fatty acids as components, a ratio of medium chain fatty acids in the total fatty acids to be produced, a ratio of lipids containing medium chain fatty acids as components in the total lipids to be produced) is significantly improved. Moreover, in the transformant, in comparison with a host, the fatty acid composition in the lipid (a ratio of specific fatty acids relative to the total tatty acids to be produced, a ratio of lipids containing specific fatty acids as components in the total lipids to be produced) changes. Therefore, the present invention using the transformant can be preferably applied to production of specific lipids, particularly medium chain fatty acids or lipids containing these medium chain fatty acids as components, preferably fatty acids having 8 or more and 16 or less carbon atoms or lipids containing these fatty acids as components, more preferably fatty acids having 8 or more and 14 or less carbon atoms or lipids containing these fatty acids as components, further preferably fatty acids having 10 or more and 14 or less carbon atoms or lipids containing these fatty acids as components, furthermore preferably fatty acids having 12 or more and 14 or less carbon atoms or lipids containing these fatty acids as components, furthermore preferably fatty acids having 12 or 14 carbon atoms or lipids containing these fatty acids as components, and furthermore preferably fatty acids having 12 carbon atoms or lipids containing these fatty acids as components.

Further, in the transformant of the embodiment, in comparison with a host itself, production efficiency of medium chain fatty acids or lipids containing these medium chain fatty acids as components is significantly improved. Therefore, the present invention using the transformant can be preferably applied to production of the lipid.

The ability to produce fatty acids and lipids of the acyl-ACP thioesterase can be measured by the method used in the Examples. Moreover, in the present specification, a cell in which the expression of a gene encoding an objective protein herein is promoted is also referred to as the “transformant”, and a cell in which the expression of the gene encoding the objective protein is not promoted is also referred to as the “host” or “wild type strain”.

A method of promoting the expression of the acyl-ACP thioesterase gene can be appropriately selected from an ordinarily method. For example, a method of introducing the acyl-ACP thioesterase gene into a host, and a method of modifying expression regulation regions of the gene (promoter, terminator, or the like) in a host having the acyl-ACP thioesterase gene on a genome, can be selected.

The method of introducing an acyl-ACP thioesterase gene into a host and promoting the expression of the gene is described.

The transformant that can be preferably used in the present invention is obtained by introducing a gene that encodes acyl-ACP thioesterase into a host according to an ordinarily genetic engineering method. Specifically, the transformant can be produced by preparing an expression vector or a gene expression cassette which is capable of expressing a gene that encodes acyl-ACP thioesterase in a host cell, introducing this vector or cassette into host cells, and thereby transforming the host cells.

The host for the transformant is not particularly limited, and can be appropriately selected from ordinarily used hosts. For example, microorganisms (including algae and microalgae), plants or animals can be used. Among these, microorganisms or plants are preferable, and microorganisms are more preferable as a host, from the viewpoints of production efficiency and the usability of lipids to be obtained.

As the microorganisms, prokaryotes and eukaryotes can be used. Prokaryotes include microorganisms belonging to the genus Escherichia, microorganisms belonging to the genus Bacillus, microorganisms belonging to the genus Synechocystis, microorganisms belonging to the genus Synechccoccus, or the like. Eukaryotes include eukaryotic microorganisms belonging to yeast, filamentous fungi or the like. Among these, from a viewpoint of the productivity of lipids, Escherichia coli belonging to the genus Escherichia, Bacillus subtilis belonging to the genus Bacillus, Rhodosporidium toruloides belonging to yeast, and Mortierella sp. belonging to filamentous fungi are preferable, and Escherichia coli is more preferable.

As the algae or microalgae, from a viewpoint o establishment of a gene recombination technique, algae belonging to the genus Chlamydomonas, algae belonging to the genus Chlorella, algae belonging to the genus Phaeodactylum, or algae belonging to the genus Nannochloropsis are preferable, and algae belonging to the genus Nannochloropsis are more preferable. Specific examples of the algae belonging to the genus Nannochloropsis include Nannochloropsis oculata, Nannochloropsis gaditana, Nannochloropsis salina, Nannochloropsis oceanica, Nannochloropsis atomus, Nannochloropsis maculata, Nannochloropsis granulata, and Nannochloropsis sp. Among these, from a viewpoint of the productivity of lipids, Nannochloropsis oculata or Nannochloropsis gaditana is preferable, and Nannochloropsis oculata is more preferable.

As the plants, from a viewpoint of a high lipid content of seeds, Arabidopsis thaliana, rapeseed, Cocos nucifera, palm, cuphea, or Jatropha curcas is preferable, and Arabidopsis thaliana is more preferable.

A vector for use as the plasmid vector for gene expression or the gene expression cassette (plasmid) may be any vector capable of introducing the gene encoding the acyl-ACP thioesterase into a host, and expressing the gene in the host cell. For example, a vector which has expression regulation regions such as a promoter and a terminator in accordance with the type of the host to be introduced, and has a replication initiation point, a selection marker or the like, can be used. Furthermore, the vector may also be a vector such as a plasmid capable of self-proliferation and self-replication outside the chromosome, or may also be a vector which is incorporated into the chromosome.

Specific examples of the vector include, in the case of using a microorganism as the host, pBluescript (pBS) II SK(−) (manufactured by Stratagene), a pSTV-based vector (manufactured by Takara Bio), pUC-based vector (manufactured by Takara Shuzo), a pET-based vector (manufactured by Takara Bio), a pGEX-based vector (manufactured by GE Healthcare), a pCold-based vector (manufactured by Takara Bio), pHY300PLK (manufactured by Takara Bio), pUB110 (Mckenzie, T. et al., (1986), Plasmid 15(2); p, 93-103), pBR322 (manufactured by Takara Bio), pRS403 (manufactured by Stratagene), and pMW218/219 (manufactured by Nippon Gene). In particular, in the case of using Escherichia coli as the host, pBluescript II SK(−) or pMW218/219 is preferably used.

When the algae are used as the host, specific examples of the vector include pUC19 (manufactured by Takara Bio), P88 (Chlamydomonas Center), P-322 (Chlamydomonas Center), pPha-T1 (see Yangmin Gong, et al., Journal of Basic Microbiology, 2011, vol. 51, p. 666-872) and pJET1 (manufactured by COSMO BIO). In particular, in the case of using the algae belonging to the genus Nannochloropsis as the host, pUC19, pPha-T1 or pJET1 is preferably used. Moreover, when the host is the algae belonging to the genus Nannochloropsis, the host can be transformed, with referring to the method described in Oliver Kilian, et al., Proceedings of the National Academy of Sciences of the United States of America , 2011; vol 108(52), by using the DNA fragment consisting of the gene of the present invention, a promoter and a terminator (gene expression cassette). Specific examples of this DNA fragment include a PCR-amplified DNA fragment and a restriction enzyme-cut DNA fragment.

In the case of using a plant cell as the host, examples of the vector include a pRI-based vector (manufactured by Takara Bio), a pBI-based vector (manufactured by Clontech), and an IN3based vector (manufactured by Inplanta Innovations). In particular, in the case of using Arabidopsis thaliana as the host, a pRI-based vector or a pBI-based vector is preferably used.

Moreover, a kind of promoter or terminator regulating the expression of the gene encoding an objective protein can also be appropriately selected according to a kind of the host to be used. Specific examples of the promoter that can be preferably used in the present invention include lac promoter, trp promoter, tac promoter, trc promoter, T7 promoter, SpoVG promoter, a promoter that relates to a derivative that can be derived by addition of isopropyl β-D-1-thiogalactopyranoside (IPTG), Rubisco operon (rbc), PSI reaction center protein (psaAB), D1 protein of PSII (psbA), cauliflower mosaic virus 35S RNA promoter, promoters for housekeeping genes (e.g., tubulin promoter, actin promoter and ubiquitin promoter), rapeseed-derived Napin gene promoter, plant-derived Rubisco promoter, a promoter of a violaxanthin/(chlorophyll a)-binding protein VCP1 gene derived from the genus Nannochloropsis (VCP1 promoter, VCP2 promoter) (WO 2014/103930), and a promoter of a oleosin-like protein LDSP (lipid droplet surface protein) gene derived from the genus Nannochloropsis (Astrid Vieler, et al., PLOS Genetics, 2012; vol. 8(11); e1003084, DOI: 10.1371).

Moreover, a kind of selection marker for confirming introduction of the gene encoding an objective protein can also be appropriately selected according to a kind of the host to be used. Examples of the selection marker that can be preferably used in the present invention include drug resistance genes such as an ampicillin resistance gene, a chloramphenicol resistance gene, an erythromycin resistance gene, a neomycin resistance gene, a kanamycin resistance gene, a spectinomycin resistance gene, a tetracycline resistance gene, a blasticidin S resistance gene, a bialaphos resistance gene, a zeocin resistance gene, a paromomycin resistance gene, and a hygromycin resistance gene. Further, it is also possible to use a deletion of an auxotrophy-related gene or the like as the selection marker gene.

Introduction of the gene encoding an objective protein to the vector can be constructed by an ordinary technique such as restriction enzyme treatment and ligation. Moreover, upon construction of the expression vector, in addition to the gene encoding the acyl-ACP thioesterase, a sequence useful for translation of the gene, for example, the sequence corresponding to the initiation codon or the termination codon can be appropriately supplemented.

The method for transformation is not particularly limited as long as it is a method capable of introducing a target gene into a host. For example, a method of using calcium ion, a general competent cell transformation method (J. Bacterial. 93, 1925 (1967)), a protoplast transformation method (Mol. Gen Genet. 168, 111 (1979)), an electroporation method (FEMS Microbiol. Lett. 55, 135 (1990)), or an LP transformation method (T. Akamatsu, et al., Archives of Microbiology, 1987, 146, p. 353-357; T. Akamatsu, et al., Bioscience, Biotechnology, and Biochemistry, 2001, 65, 4, p. 823-829), can be used. When the host is the algae belonging to the genus Nannochloropsis, transformation can also be performed by using the electroporation method described in Randor Radakovits, et al., Nature Communications, DOI: 10.1038/ncomms 1688, 2012.

The selection of a transformant having a target gene fragment introduced therein can be carried out by utilizing the selection marker or the like. For example, the selection can be carried out by using an indicator whether a transformant acquires the drug resistance as a result of introducing a drug resistance gene derived from a vector into a host cell together with a target DNA fragment upon the transformation. Further, the introduction of a target DNA fragment can also be confirmed by PCR using a genome as a template and the like.

In a host having an acyl-ACP thioesterase gene on a genome, a method of modifying expression regulation regions of the gene and promoting the expression of the gene is described.

The “expression regulation region” indicates the promoter or the terminator, in which these sequences are generally involved in regulation of the expression amount (transcription amount, translation amount) of the gene adjacent thereto. In a host having the above-described acyl-ACP thioesterase gene on a genome, productivity of medium chain fatty acids or lipids containing these medium chain fatty acids as components can be improved by modifying expression regulation regions of the gene and promoting the expression of the acyl-ACP thioesterase gene.

Specific examples of the method of modifying the expression regulation region include interchange of promoters. In the host having the above-mentioned acyl-ACP thioesterase gene on the genome, the expression of the above-described acyl-ACP thioesterase gene can be promoted by interchanging the promoter of the gene (hereinafter, also referred to as “acyl-ACP thioesterase promoter”) with a promoter having higher transcriptional activity.

The promoter used for interchanging the acyl-ACP thioesterase promoters is not particularly limited, and can be appropriately selected from the promoters that are higher in the transcriptional activity than the acyl-ACP thioesterase promoter and suitable for production of the medium chain fatty acids or the lipids containing these fatty acids as the components.

The above-described modification of a promoter can employ according to an ordinarily method such as homologous recombination. Specifically, a linear DNA fragment containing an upstream and downstream regions of a target promoter and containing other promoter instead of the target promoter is constructed, and the resultant DNA fragment is incorporated into a host cell to cause double crossover homologous recombination on the side upstream and downstream of the target promoter of the host genome. Then the target promoter on the genome is substituted with other promoter fragment, and the promoter can be modified.

The method of modifying a target promoter according to such homologous recombination can be conducted with, for example, reference to literature such as Besher et al., Methods in molecular biology, 1995, vol. 47, p. 291-302.

### 4. Method of Producing Lipid

In the transformant of the present invention, productivity of the medium chain fatty acids or the lipids containing these fatty acids as the components is improved in comparison with the host. Accordingly, if the transformant of the present invention is cultured under suitable conditions and then the medium chain fatty acids or the lipids containing these fatty acids as the components are collected from a cultured product obtained, the medium chain fatty acids or the lipids containing these fatty acids as the components can be efficiently produced.

From a viewpoint of improvement in the productivity of lipids, the method of producing a lipid of the present invention preferably includes a step of obtaining a cultured product by culturing, under suitable conditions, the transformant having the introduced gene encoding the acyl-ACP thioesterase; and a step of collecting the lipid from the resulting cultured product.

In addition, an expression “culture the transformant” described in the present invention means culturing or growing of the microorganisms, the algae, the plants or the animals, or cells or tissues thereof, including cultivating of the plants in soil or the like. Herein, the “cultured product” includes a transformant itself subjected to cultivation or the like, in addition to the medium used for culture.

The culture condition can be appropriately selected in accordance with the type of the host for transformant, and any ordinary used culture condition can be employed.

Further, from a viewpoint of the production efficiency of lipids, substrates of acyl-ACP thioesterase or precursor substances participating in the fatty acid biosynthesis system, such as glycerol, acetic add or malonic acid, may be added to the medium.

For example, in the case of using Escherichia coli as the host for transformant, culture may be carried out in LB medium or Overnight Express Instant TB Medium (manufactured by Novagen) at 30° C. to 37° C. for half a day to 1 day.

In the case of using Arabidopsis thaliana as the host for transformant, growth may be carried out at soil under the temperature conditions of 20° C. to 25° C., by continuously irradiating white light or under light illumination conditions of a light period of 16 hours and a dark period of 8 hours, for one to two months.

When the host of the transformant is the algae, medium based on natural seawater or artificial seawater may be used. Alternatively, commercially available culture medium may also be used. Specific examples of the culture medium include f/2 medium, ESM medium, Daigo IMK medium, L1 medium and MNK medium. Above all, from viewpoints of an improvement in the productivity of lipids and a nutritional ingredient concentration, f/2 medium, ESM medium or Daigo IMK medium is preferred; f/2 medium or Daigo IMK medium is more preferred; and f/2 medium is further preferred. For growth promotion of the algae and an improvement in productivity of fatty acids, a nitrogen source, a phosphorus source, metal salts, vitamins, trace metals or the like can be appropriately added lo the culture medium. An amount of the algae to be seeded to the culture medium is not particularly limited. In view of viability, the amount is preferably 1% to 50% (vol/vol), and more preferably 1% to 10% (vol/vol), per culture medium. Culture temperature is not particularly limited within the range in which the temperature does not adversely affect growth of the algae, and is ordinarily in the range of 5° C. to 40° C. From viewpoints of the growth promotion of the algae, the improvement in productivity of fatty acids, and reduction of production cost, the temperature is preferably 10° C. to 35° C., and more preferably 15° C. to 30° C.

Moreover, the algae are preferably cultured under irradiation with light so that photosynthesis can be made. The light irradiation only needs to be made under conditions in which the photosynthesis can be made, and artificial light or sunlight may be applied. From viewpoints of the growth promotion of the algae and the improvement in the productivity of fatty acids, irradiance during the light irradiation is preferably in the range of 100 lx to 50,000 lx, more preferably in the range of 300 lx to 10,000 lx, and further preferably 1,000 lx to 6,000 lx. Moreover, an interval of the light irradiation is not particularly limited. From the viewpoints in a manner similar to the viewpoints described above, the irradiation is preferably performed under a light and dark cycle. In 24 hours, a light period is preferably from 8 to 24 hours, more preferably from 10 to 18 hours, and further preferably 12 hours.

Moreover, the algae are preferably cultured in the presence of a carbon dioxide-containing gas or in a culture medium containing carbonate such as sodium hydrogen carbonate so that the photosynthesis can be made. A concentration of carbon dioxide in the gas is not particularly limited. From viewpoints of the growth promotion and the improvement in the productivity of fatty acids, the concentration is preferably from 0.03% (which is the same degree as the concentration under atmospheric conditions) to 10%, more preferably from 0.05% to 5%, further preferably from 0.1% to 3%, and furthermore preferably from 0.3% to 1%. A concentration of the carbonate is not particularly limited. When the sodium hydrogen carbonate is used, for example, from viewpoints of the growth promotion and the improvement in the productivity of fatty acids, the concentration is preferably from 0.01% to 5% by mass, more preferably from 0.05% to 2% by mass, and further preferably from 0.1% to 1% by mass.

A culture time is not particularly limited, and the culture may be performed for a long time (for example, about 150 days) so that an alga body in which the lipid is accumulated at a high concentration can grow at a high concentration. From viewpoints of the growth promotion of the algae, the improvement in the productivity of fatty acids, and the reduction of production cost, the culture time is preferably from 3 to 90 days, more preferably from 3 to 30 days, and further preferably from 7 to 30 days. The culture may be performed in any of aerated and agitated culture, shaking culture or static culture. From a viewpoint of improving air-permeability, aerated and agitated culture is preferred.

Lipids produced in the transformant is collected by an ordinary method used for isolating lipid components and the like contained in the living body of the transformant. For example, lipid components can be isolated and collected from the above-described cultured product or the transformant by means of filtration, centrifugation, cell disruption, gel filtration chromatography, ion exchange chromatography, chloroform/methanol extraction, hexane extraction, or ethanol extraction. In the case of isolation and collection of larger scales, lipids can be obtained by collecting oil components from the cultured product or the transformant through pressing or extraction, and then performing general purification processes such as degumming, deacidification, decoloration, dewaxing, and deodorization. After lipid components are isolated as such, the isolated lipids are hydrolyzed, and thereby fatty acids can be obtained. Specific examples of the method of isolating fatty acids from lipid components include a method of treating the lipid components at a high temperature of about 70° C. in an alkaline solution, a method of performing a lipase treatment, and a method of degrading the lipid components using high-pressure hot water.

In the acyl-ACP thioesterase of the present invention, specificity to the medium chain acyl-ACP or C12 to C16 acyl-ACP, particularly C12 acyl-ACP or C14 acyl-ACP is high. In the transformant of the present invention, the ratio of the content of medium chain fatty acids, for example, fatty acids having 8 to 16 carbon atoms, preferably fatty acids having 8 to 14 carbon atoms, more preferably fatty acids having 10 to 14 carbon atoms, further preferably fatty acids having 12 to 14 carbon atoms, furthermore preferably fatty acids having 12 or 14 carbon atoms, and furthermore preferably fatty acids having 12 carbon atoms each in the total fatty acid components increases. Therefore, the production method in which the transformant is used of the present invention can be preferably applied to production of lipids, particularly medium chain fatty acids, preferably fatty acids having 8 to 16 carbon atoms, more preferably fatty acids having 8 to 14 carbon atoms, further preferably fatty acids having 10 to 14 carbon atoms, furthermore preferably fatty acids having 12 to 14 carbon atoms, furthermore preferably fatty acids having 12 or 14 carbon atoms, and furthermore preferably fatty acids having 12 carbon atoms or a lipids containing these fatty acids as components.

The lipids produced in the production method of the present invention preferably contain fatty acids or fatty acid compounds, and more preferably contain fatty acids or fatty acid ester compounds thereof, in view of usability thereof. Specifically, the lipids produced in the production method of the present invention preferably contain fatty acids having 8 or more and 16 or less carbon atoms or fatty acid ester compounds thereof, more preferably fatty acids having 8 or more and 14 or less carbon atoms or fatty acid ester compounds thereof, further preferably fatty acids having 10 or more and 14 or less carbon atoms or fatty acid ester compounds thereof, furthermore preferably fatty acids having 12 or more and 14 or less carbon atoms or fatty acid ester compounds thereof, furthermore preferably fatty acids having 12 or 14 carbon atoms or fatty acid ester compounds thereof, and furthermore preferably fatty acids having 12 carbon atoms or fatty acid ester compounds thereof. From usability for a surfactant or the like, the fatty acid or the fatty acid ester compound thereof contained in the lipid is preferably a fatty acid having 8 to 16 carbon atoms or a fatty acid ester compound thereof, more preferably a fatty acid having 8 to 14 carbon atoms or a fatty acid ester compound thereof, further preferably a fatty acid having 10 to 14 carbon atoms or a fatty acid ester compound thereof, furthermore preferably a fatty acid having 12 to 14 carbon atoms or a fatty acid ester compound thereof, furthermore preferably a fatty acid having 12 or 14 carbon atoms or a fatty acid ester compound thereof, and furthermore preferably a fatty acid having 12 carbon atoms or a fatty acid ester compound thereof.

From a viewpoint of the productivity, the fatty acid ester compound is preferably a simple lipid or a complex lipid, more preferably a simple lipid, and further preferably a triacylglycerol.

The fatty acids and lipids obtained by the production method or the transformant of the present invention can be utilized for food, as well as an emulsifier incorporated into cosmetic products or the like, a cleansing agent such as a soap or a detergent, a fiber treatment agent, a hair conditioning agent, a disinfectant or an antiseptic.

With regard to the embodiments described above, the present invention also discloses methods, transformants, proteins, and genes described below.

<1> A method of producing a lipid, containing the steps of:

culturing a transformant in which a gene encoding any one of the following proteins (A) to (C) is introduced into a host, and

collecting a lipid from the cultured product:

(A) a protein consisting of the amino acid sequence of the 611th to 772nd positions set forth in SEQ ID NO: 1;
(B) a protein consisting of an amino acid sequence having 80% or more identity with the amino acid sequence of the 611th to 772nd positions set forth in SEQ ID NO: 1, and having acyl-ACP thioesterase activity; and
(C) a protein containing the amino acid sequence of the protein (A) or (B), and having acyl-ACP thioesterase activity.
<2> A method of enhancing productivity of fatty acids or a lipid containing the fatty acids as components produced in a cell of a transformant, containing the step of introducing a gene encoding any one of the proteins (A) to (C) into a host.
<3> The method described in the above item <2>, wherein the hold is a medium chain fatty acid or a lipid containing the medium chain fatty acids as components.
<4> A method of modifying the composition of a lipid, containing the steps of:

introducing a gene encoding any one of the proteins (A) to (C) into a host, and thereby obtaining a transformant, and

enhancing productivity of medium chain fatty acids or a lipid containing the fatty acids as components produced in a cell of the transformant, to modify the composition of fatty acids or a lipid in all fatty acids or all lipids to be produced.

<5> A method of producing a lipid, containing the steps of:

culturing a transformant in which the expression of a gene encoding any one of the proteins (A) to (C) is enhanced, and

collecting a lipid from the cultured product.

<6> A method of enhancing productivity of fatty acids or a lipid containing the fatty acids as components produced in a cell of a transformant, containing the step of enhancing the expression of a gene encoding any one of the proteins (A) to (C).
<7> The method described in the above item <6>, wherein the lipid is medium chain fatty acids or a lipid containing the medium chain fatty acids as components.
<8> A method of modifying the composition of a lipid, containing the steps of:

enhancing the expression of a gene encoding any one of the proteins (A) to (C), and

enhancing productivity of medium chain fatty acids or a lipid containing the fatty acids as components produced in a cell of a transformant, to modify the composition of fatty acids or a lipid in all fatty acids or all lipids to be produced.

<9> The method described in any one of the above items <5> to <9>, containing the steps of introducing a gene encoding any one of the proteins (A) to (C) into a host, and enhancing the expression of the gene.
<10> The method described in any one of the above items <1> to <9>, wherein the identity of the protein (B) with the amino acid sequence of the 611th to 772nd positions set forth in SEQ ID NO: 1 is 85% or more, preferably 90% or more, more preferably 95% or more, further preferably 96% or more, furthermore preferably 97% or more, furthermore preferably 98% or more, and furthermore preferably 99% or more.
<11> The method described in any one of the above items <1> to <10>, wherein the protein (B) consists of an amino acid sequence in which 1 or several amino acids, preferably 1 or more and 20 or less amino acids, more preferably 1 or more and 15 or less amino acids, further preferably 1 or more and 10 or less amino acids, furthermore preferably 1 or more and 8 or less amino acids, furthermore preferably 1 or more and 5 or less amino acids, furthermore preferably 1 or more and 4 or less amino acids, furthermore preferably 1 or more and 3 or less amino adds, and furthermore preferably 1 or 2 amino acids, are deleted, substituted, inserted or added to the amino acid sequence of the 611th to 772nd positions set forth in SEQ ID NO: 1; and has acyl-ACP thioesterase activity.
<12> The method described in any one of the above items <1> to <9>, wherein the protein (C) consists of an amino add sequence in which an amino acid on an N-terminal side is deleted at an arbitrary position of the 1st to 610th positions set forth in SEQ ID NO: 1.
<13> The method described in any one of the above items <1> to <9>, wherein the protein (C) is any one of the following proteins (C1) to (C20):
(C1) a protein consisting of the amino acid sequence of the 1st to 772nd positions set forth in SEQ ID NO: 1;
(C2) a protein consisting of the amino acid sequence of the 487th to 772nd positions set forth in SEQ ID NO: 1;
(C3) a protein consisting of the amino acid sequence of the 488th to 772nd positions set forth in SEQ ID NO: 1;
(C4) a protein consisting of the amino acid sequence of the 497th to 772nd positions set forth in SEQ ID NO: 1;
(C5) a protein consisting of the amino acid sequence of the 507th to 772nd positions set forth in SEQ ID NO: 1;
(C6) a protein consisting of the amino acid sequence of the 517th to 772nd positions set forth in SEQ ID NO: 1;
(C7) a protein consisting of the amino acid sequence of the 527th to 772nd positions set forth in SEQ ID NO: 1;
(C8) a protein consisting of the amino acid sequence of the 537th to 772nd positions set forth in SEQ ID NO: 1;
(C9) a protein consisting of the amino acid sequence of the 547th to 772nd positions set forth in SEQ ID NO: 1;
(C10) a protein consisting of the amino acid sequence of the 557th to 772nd positions set forth in SEQ ID NO: 1;
(C11) a protein consisting of the amino acid sequence of the 567th to 772nd positions set forth in SEQ ID NO: 1;
(C12) a protein consisting of the amino acid sequence of the 577th to 772nd positions set forth in SEQ ID NO: 1;
(C13) a protein consisting of the amino acid sequence of the 587th to 772nd positions set forth in SEQ ID NO: 1;
(C14) a protein consisting of the amino acid sequence of the 597th to 772nd positions set forth in SEQ ID NO: 1;
(C15) a protein consisting of the amino acid sequence of the 607th to 772nd positions set forth in SEQ ID NO: 1;
(C16) a protein consisting of the amino acid sequence of the 608th to 772nd positions set forth in SEQ ID NO: 1;
(C17) a protein consisting of the amino acid sequence of the 609th to 772nd positions set: forth in SEQ ID NO: 1;
(C18) a protein consisting of the amino acid sequence of the 610th to 772nd positions set forth in SEQ ID NO: 1;
(C19) a protean consisting of an amino acid sequence having 80% or more identity, preferably 85% or more identity, more preferably 90% or more identity, further preferably 95% or more identity, furthermore preferably 96% or more identity, furthermore preferably 97% or more identity, furthermore preferably 98% or more identity, and furthermore preferably 99% or more identity, with the amino acid sequence of any one of the proteins (C1) to (C18), and having acyl-ACP thioesterase activity; and
(C20) a protein consisting of an amino acid sequence in which 1 or several amino acids, preferably 1 or more and 20 or less amino acids, more preferably 1 or more and 15 or less amino acids, further preferably 1 or more and 10 or less amino acids, furthermore preferably 1 or more and 8 or less amino acids, furthermore preferably 1 or more and 5 or less amino acids, furthermore preferably 1 or more and 4 or less amino acids, furthermore preferably 1 or more and 3 or less amino acids, and furthermore preferably 1 or 2 amino acids, are deleted, substituted, inserted or added to the amino acid sequence of any one of the proteins (C1) to (C18), and having acyl-ACP thioesterase activity.
<14> The method described in any one of the above items <1> to <13>, wherein the gene encoding any one of the proteins (A) to (C) is a gene consisting of any one of the following DNAs (a) to (f);
(a) a DNA consisting of the nucleotide sequence of the 1,831st to 2,319th positions set forth in SEQ ID NO: 2;
(b) a DNA consisting of a nucleotide sequence having 80% or more identity, preferably 85% or more identity, more preferably 90% or more identify, further preferably 95% or more identity, furthermore preferably 96% or more identity, furthermore preferably 97% or more identity, furthermore preferably 98% or more identity, and furthermore preferably 99% or more identity, with the nucleotide sequence of the 1,831st to 2,319th positions set forth in SEQ ID NO: 2, and encoding a protein having acyl-ACP thioesterase activity;
(c) a DNA containing the nucleotide sequence of the DNA (a) or (b), and encoding a protein having acyl-ACP thioesterase activity;
(d) a DNA consisting of the nucleotide sequence of the 373rd to 861st positions set forth in SEQ ID NO: 3;
(e) a DNA consisting of a nucleotide sequence having 80% or more identity, preferably 85% or more identity, more preferably 90% or more identity, further preferably 95% or more identity, furthermore preferably 96% or more identity, furthermore preferably 97% or more identity, furthermore preferably 98% or more identity, and furthermore preferably 99% or more identity, with the nucleotide sequence of the 373rd to 861st positions set forth in SEQ ID NO: 3, and encoding a protein having acyl-ACP thioesterase activity; and
(f) a DNA containing the nucleotide sequence of the DNA (d) or (e), and encoding a protein having acyl-ACP thioesterase activity.
<15> The method described in the above item <14>, wherein the DNA (b) is a DNA consisting of a nucleotide sequence in which 1 or several nucleotides, preferably 1 or more and 20 or less nucleotides, more preferably 1 or more and 15 or less nucleotides, further preferably 1 or more and 10 or less nucleotides, furthermore preferably 1 or more and 8 or less nucleotides, furthermore preferably 1 or more and 5 or less nucleotides, furthermore preferably 1 or more and 4 or less nucleotides, furthermore preferably 1 or more and 3 or less nucleotides, and furthermore preferably 1 or 2 nucleotides, are deleted, substituted, inserted or added to the nucleotide sequence of DNA (a); and encoding a protein having acyl-ACP thioesterase activity, or a DNA capable of hybridizing with a DNA consisting of a nucleotide sequence complementary with the DNA (a) under a stringent condition, and encoding a protein having acyl-ACP thioesterase activity.
<16> The method described in the above item <14>, wherein the DNA (c) consists of a nucleotide sequence in which nucleotides on a 5′-terminal side are deleted at an arbitrary position of the 1st to 1,830th positions set forth in SEQ ID NO: 2.
<17> The method described in the above item <14>, wherein the DNA (c) is any one of the following DNAs (c1) to (c20):
(c1) a DNA consisting of the nucleotide sequence of the 1st to 2,319th positions set forth in SEQ ID NO: 2;
(c2) a DNA consisting of the nucleotide sequence of the 1,459th to 2,319th positions set forth in SEQ ID NO: 2;
(c3) a DNA consisting of the nucleotide sequence of the 1,462nd to 2,319th positions set forth in SEQ: ID NO: 2;
(c4) a DNA consisting of the nucleotide sequence of the 1,489th to 2,319th positions set forth in SEQ ID NO: 2;
(c5) a DNA consisting of the nucleotide sequence of the 1,519th to 2,319th positions set forth in SEQ ID NO: 2;
(c6) a DNA consisting of the nucleotide sequence of the 1,540th to 2,319th positions set forth in SEQ ID NO: 2;
(c7) a DNA consisting of the nucleotide sequence of the 1,579th to 2,319th positions set forth in SEQ ID NO: 2;
(c8) a DNA consisting of the nucleotide sequence of the 1,609th to 2,319th positions set forth in SEQ ID NO: 2;
(c9) a DNA consisting of the nucleotide sequence of the 1,639th to 2,319th positions set forth in SEQ ID NO: 2;
(c10) a DNA consisting of the nucleotide sequence of the 1,689th to 2,319th positions set forth in SEQ ID NO: 2;
(c11) a DNA consisting of the nucleotide sequence of the 1,699th to 2,319th positions set forth in SEQ ID NO: 2;
(c12) a DNA consisting of the nucleotide sequence of the 1,729th to 2,319th positions set forth in SEQ ID NO: 2;
(c13) a DNA consisting of the nucleotide sequence of the 1,759th to 2,319th positions set forth in SEQ ID NO: 2;
(c14) a DNA consisting of the nucleotide sequence of the 1,789th to 2,319th positions set forth in SEQ ID NO: 2;
(c15) a DNA consisting of the nucleotide sequence of the 1,819th to 2,319th positions set forth in SEQ ID NO: 2;
(c16) a DNA consisting of the nucleotide sequence of the 1,822nd to 2,313th positions set forth in SEQ ID NO: 2;
(c17) a DNA consisting of the nucleotide sequence of the 1,825th to 2,319th positions set forth in SEQ ID NO: 2;
(c18) a DNA consisting of the nucleotide sequence of the 1,828th to 2,319th positions set forth in SEQ ID NO: 2;
(c19) a DNA consisting of a nucleotide sequence having 80% or more identity, preferably 85% or more identity, more preferably 90% or more identity, further preferably 95% or more identity, furthermore preferably 96% or more identity, furthermore preferably 97% or more identity, furthermore preferably 98% or more identity, and furthermore preferably 99% or more identity, with the nucleotide sequence of any one of the DNAs (c1) to (c18), and encoding a protein having acyl-ACP thioesterase activity, and
(c20) a DNA consisting of a nucleotide sequence in which 1 or several nucleotides, preferably 1 or more and 20 or less nucleotides, more preferably 1 or more and 15 or less nucleotides, further preferably 1 or more and 10 or less nucleotides, furthermore preferably 1 or more and 8 or less nucleotides, furthermore preferably 1 or more and 5 or less nucleotides, furthermore preferably 1 or more and 4 or less nucleotides, furthermore preferably 1 or more and 3 or less nucleotides, and furthermore preferably 1 or 2 nucleotides, are deleted, substituted, inserted or added to the nucleotide sequence of any one of the DNAs (c1) to (c18), and encoding a protein having acyl-ACP thioesterase activity.
<18> The method described in the above item <14>, wherein the DNA (e) is a DNA consisting of a nucleotide sequence in which 1 or several nucleotides, preferably 1 or more and 20 or less nucleotides, more preferably 1 or more and 15 or less nucleotides, further preferably 1 or more and 10 or less nucleotides, furthermore preferably 1 or more and 8 or less nucleotides, furthermore preferably 1 or more and 5 or less nucleotides, furthermore preferably 1 or more and 4 or less nucleotides, furthermore preferably 1 or more and 3 or less nucleotides, and furthermore preferably 1 or 2 nucleotides, are deleted, substituted, inserted or added to the nucleotide sequence of the DNA (d), and encoding a protein having acyl-ACP thioesterase activity, or a DNA capable of hybridizing with a DNA consisting of a nucleotide sequence complementary with the DNA (d) under a stringent condition, and encoding a protein having acyl-ACP thioesterase activity.
<19> The method described in the above item <14>, wherein the DNA (f) consists of a nucleotide sequence in which nucleotides on a 5′-terminal side are deleted at an arbitrary position of the 1st to 372nd positions set forth in SEQ ID NO: 3.
<20> The method described in the above item <14> wherein the DNA (f) is any one of the following DNAs (f1) to (f19):
(f1) a DNA consisting of the nucleotide sequence of the 1st to 861st positions set forth in SEQ ID NO: 3;
(f2) a DNA consisting of the nucleotide sequence of the 4th to 861st positions set forth in SEQ ID NO: 3;
(f3) a DNA consisting of the nucleotide sequence of the 31st to 861st positions set forth in SEQ ID NO: 3;
(f4) a DNA consisting of the nucleotide sequence of the 61st to 861st positions set forth in SEQ ID NO: 3;
(f5) a DNA consisting of the nucleotide sequence of the 91st to 861st positions set forth in SEQ ID NO: 3;
(f6) a DNA consisting of the nucleotide sequence of the 121st to 861st positions set forth in SEQ ID NO: 3;
(f7) a DNA consisting of the nucleotide sequence of the 151st to 861st positions set forth in SEQ ID NO: 3;
(f8) a DNA consisting of the nucleotide sequence of the 181st to 861st positions set forth in SEQ ID NO: 3;
(f9) a DNA consisting of the nucleotide sequence of the 211th to 861st positions set forth in SEQ ID NO: 3;

(f10) a DNA consisting of the nucleotide sequence of the 241st to 861st positions set forth in SEQ ID NO: 3;

(f11) a DNA consisting of the nucleotide sequence of the 271st to 861st positions set forth in SEQ ID NO: 3;
(f12) a DNA consisting of the nucleotide sequence of the 301st to 861st positions set forth in SEQ ID NO: 3;
(f13) a DNA consisting of the nucleotide sequence of the 331st to 861st positions set forth in SEQ ID NO: 3;
(f14) a DNA consisting of the nucleotide sequence of the 361st to 861st positions set forth in SEQ ID NO: 3;
(f15) a DNA consisting of the nucleotide sequence of the 384th to 861st positions set forth in SEQ ID NO: 3;
(f16) a DNA consisting of the nucleotide sequence of the 367th to 861st positions set forth in SEQ ID NO: 3;
(f17) a DNA consisting of the nucleotide sequence of the 370th to 861st positions set forth in SEQ ID NO: 3;
(f18) a DNA consisting of a nucleotide sequence having 80% or more identity, preferably 85% or more identity, more preferably 90% or more identity, further preferably 95% or more identity, furthermore preferably 96% or more identity, furthermore preferably 97% or more identity, furthermore preferably 98% or more identity, and furthermore preferably 99% or more identity, with the nucleotide sequence of any one of the DNAs (f1) to (f17), and encoding a protein having acyl-ACP thioesterase activity; and
(f19) a DNA consisting of a nucleotide sequence in which 1 or several nucleotides, preferably 1 or more and 20 or less nucleotides, more preferably 1 or more and 15 or less nucleotides, further preferably 1 or more and 10 or less nucleotides, furthermore preferably 1 or more and 8 or less nucleotides, furthermore preferably 1 or more and 5 or less nucleotides, furthermore preferably 1 or more and 4 or less nucleotides, furthermore preferably 1 or more and 3 or less nucleotides, and furthermore preferably 1 or 2 nucleotides, are deleted, substituted, inserted or added to the nucleotide sequence of any one of the DNAs (f1) to (f17), and encoding a protein having acyl-ACP thioesterase activity.
<21 > The method described in any one of the above items <1> to <20>, wherein a host of the transformant is a microorganism.
<22> The method described in the above item <21>, wherein the microorganism is Escherichia coli.
<23> The method described in the above item <21>, wherein the microorganism is a microalga.
<24> The method described in the above item <23>, wherein the microalga is an alga belonging to the genus Nannochloropsis, preferably Nannochloropsis oculata.
<25> The method described in any one of the above stems <1> to <24>, wherein the lipid contains a medium chain fatty acid or a fatty acid ester compound thereof, preferably a fatty acid having 8 or more and 16 or less carbon atoms or a fatty acid ester compound thereof, more preferably a fatty acid having 8 or more and 14 or less carbon atoms or a fatty acid ester compound thereof, further preferably a fatty acid having 10 or more and 14 or less carbon atoms or a fatty acid ester compound thereof, furthermore preferably a fatty acid having 12 or more and 14 or less carbon atoms or a fatty acid ester compound thereof, furthermore preferably a fatty add having 12 or 14 carbon atoms or a fatty acid ester compound thereof, and furthermore preferably a fatty acid having 12 carbon atoms or a fatty acid ester compound thereof.

<26> The proteins (A) to (C) specified in any one of the above items <1> to <25>.

<27> A gene encoding the protein described in the above item <26>.
<28> A gene consisting of any one of the DNAs (a) to (f) specified in any one of the above items <1> to <25>.
<29> A recombinant vector, containing the gene described in the above item <27> or <28>.
<30> A transformant, which is obtained by introducing the gene described in the above item <27> or <28> or the recombinant vector described in the above item <29> into a host.
<31> A method of producing a transformant, containing introducing the gene described in the above item <27> or <28> or the recombinant vector described in the above item <29> into a host.
<32> A transformant, wherein the expression of the gene described in the above item <27> or <28> is promoted.
<33> The transformant or the method of producing the same described in any one of the above items <30> to <32>, wherein the host of the transformant is a microorganism.
<34> The transformant or the method of producing the same described in the above item <33>, wherein the microorganism is Escherichia coli.
<35> The transformant or the method of producing the same described in the above item <33>, wherein the microorganism is a microalga.
<36> The transformant or the method of producing the same described in the above item <35>, wherein the microalga is an alga belonging to the genus Nannochloropsis, preferably Nannochloropsis oculata.
<37> Use of the protein, the gene, the recombinant vector, the transformant or a transformant obtained by the method of producing a transformant described in any one of the above items <26> to <36>, for producing a lipid.
<38> The use described in the above item <37>, wherein the lipid contains a medium chain fatty acid or a fatty acid ester compound thereof, preferably a fatty acid having 8 or more and 16 or less carbon atoms or a fatty acid ester compound thereof, more preferably a fatty acid having 8 or more and 14 or less carbon atoms or a fatty acid ester compound thereof, further preferably a fatty acid having 10 or more and 14 or less carbon atoms or a fatty acid ester compound thereof, furthermore preferably a fatty acid having 12 or more and 14 or less carbon atoms or a fatty acid ester compound thereof, furthermore preferably a fatty acid having 12 or 14 carbon atoms or a fatty acid ester compound thereof, and furthermore preferably a fatty acid having 12 carbon atoms or a fatty acid ester compound thereof.

### EXAMPLES

Hereinafter, the present invention will be described more in detail with reference to Examples, but the present invention is not limited thereto. Herein, the nucleotide sequences of the primers used in Examples are shown in Tables 1 and 2.

TABLE 1

| Primer | | SEQ ID |
|---|---|---|
| No. | Nucleotide sequence (5′ → 3′) | NO: |
| 4 | gcggccgctctagagccgctgtggtacttttgtcc | SEQ ID |
| | | NO: 4 |
| 5 | acaaaatattaacgcttattcttttttccgcggaa | SEQ ID |
| | | NO: 5 |
| 6 | ctctagagcggccgccaccg | SEQ ID |
| | | NO: 6 |
| 7 | gcgttaatattttgttaaaattcg | SEQ ID |
| | | NO: 7 |
| 8 | gcggccgctctagagcgtccgcaaggcactgcctc | SEQ ID |
| | | NO: 8 |
| 9 | gcggccgctctagagagcccaaccaaaatgcgcgg | SEQ ID |
| | | NO: 9 |
| 10 | gcggccgctctgtgtttgtttgccaagcaagcaac | SEQ ID |
| | | NO: 10 |
| 11 | gcggccgctctagagattggcggtgcagcattggc | SEQ ID |
| | | NO: 11 |
| 12 | gcggccgctctagaggttgcctgggcggaaggcta | SEQ ID |
| | | NO: 12 |
| 13 | gcggccgctctagagcactcagtggcaggcgaatg | SEQ ID |
| | | NO: 13 |
| 14 | gcggccgctctagagcgtccccactatgaggttct | SEQ ID |
| | | NO: 14 |
| 15 | gcggccgctctagagtgtttcacggccatttgcct | SEQ ID |
| | | NO: 15 |
| 16 | gcggccgctctagagctgtatctctatgtcccgaa | SEQ ID |
| | | NO: 16 |
| 17 | gcggccgctctagagccgaaaatcggcaccatcat | SEQ ID |
| | | NO: 17 |
| 18 | gcggccgctctagagttctttccgcccaccctgaa | SEQ ID |
| | | NO: 18 |
| 19 | gcggccgctctagagtgggaggaggaaattgcgtc | SEQ ID |
| | | NO: 19 |
| 20 | gcggccgctctagaggaggaggaaattgcgtctcg | SEQ ID |
| | | NO: 20 |
| 21 | gcggccgctctagaggaggaaattgcgtctcgtcc | SEQ ID |
| | | NO: 21 |
| 22 | gcggccgctctagaggaaattgcgtctcgtcctgg | SEQ ID |
| | | NO: 22 |
| 23 | gcggccgctctagagattgcgtctcgtcctggactg | SEQ ID |
| | | NO: 23 |

TABLE 2

| Pri- | | |
|---|---|---|
| mer | | SEQ ID |
| No. | Nucleotide sequence (5′ → 3′) | NO: |
| 26 | cttttttgtgaagcaatggccaagttgaccagtgccg | SEQ ID |
| | | NO: 26 |
| 27 | tttcccccatcccgattagtcctgctcctcggccac | SEQ ID |
| | | NO: 27 |
| 28 | cgagctcggtacccgactgcgcatggattgaccga | SEQ ID |
| | | NO: 28 |
| 29 | tgcttcacaaaaaagacagcttcttgat | SEQ ID |
| | | NO: 29 |
| 30 | tcgggatgggggaaaaaaacctctg | SEQ ID |
| | | NO: 30 |
| 31 | actctagaggatcccctttcgtaaataaatcagctc | SEQ ID |
| | | NO: 31 |
| 33 | gggatcctatagagtcgacc | SEQ ID |
| | | NO: 33 |
| 34 | cgggtaccgagctcgaattc | SEQ ID |
| | | NO: 34 |
| 35 | cgcggtgttgcgcgcccgctgtggtacttttgtcc | SEQ ID |
| | | NO: 35 |
| 36 | cgcggtgttgcgcgcattggcggtgcagcattggc | SEQ ID |
| | | NO: 36 |
| 37 | cgcggtgttgcgcgcccgaaaatcggcaccatcat | SEQ ID |
| | | NO: 37 |
| 38 | cgcggtgttgcgcgcttctttccgcccaccctgaa | SEQ ID |
| | | NO: 38 |
| 39 | cgcggtgttgcgcgctgggaggaggaaattgcgtc | SEQ ID |
| | | NO: 39 |
| 40 | ctcttccacagaagcttattcttttttccgcggaa | SEQ ID |
| | | NO: 40 |
| 41 | cgagctcggtacccgttcttccgcttgttgctgcc | SEQ ID |
| | | NO: 41 |
| 42 | tgttgatgcgggctgagattggtgg | SEQ ID |
| | | NO: 42 |
| 43 | cagcccgcatcaacaatgaagaccgccgctctcctc | SEQ ID |
| | | NO: 43 |
| 44 | gcgcgcaacaccgcgggtgcgggagaac | SEQ ID |
| | | NO: 44 |
| 45 | gcttctgtggaagagccagtg | SEQ ID |
| | | NO: 45 |
| 46 | ggcaagaaaagctgggggaaaagacagg | SEQ ID |
| | | NO: 46 |
| 50 | ccagcttttcttgccactgcgcatggattgaccga | SEQ ID |
| | | NO: 50 |

### Examples 1

### Preparation of Acyl-ACP Thioesterase Gene, Transformation of Escherichia coli, and Producing Lipid by Transformant

(1) Preparation of Acyl-ACP Thioesterase Gene Derived From Guillardia theta

The amino acid sequence of the protein having unknown functions derived from Guillardia theta, which is registered with the protein database of National Center for Biotechnology information (NCBI) as Accession No. XP_005824882 (SEQ ID NO: 1), and the gene sequence encoding the same (SEQ ID NO: 2) were obtained. Hereinafter, this protein is also referred to as “GtTE”, and a gene encoding the protein is also referred to as “GtTE gene”.

Subsequently, the nucleotide sequence set forth in SEQ ID NO: 3 was obtained as a nucleotide sequence subjected to codon optimization to the nucleotide sequence of the 1,459th to 2,319th positions set forth in SEQ ID NO: 2 (corresponding to the 487th to 772nd positions set forth in SEQ ID NO: 1) along with the using frequency of the codon of Escherichia coli. A gene consisting of a nucleotide sequence set forth in SEQ ID NO: 3 was obtained utilizing a custom artificial gene synthesis service provided by Operon Biotechnologies Inc.

### (2) Construction of Plasmid for GtTE Gene Expression

Using an artificially synthesized gene consisting of the nucleotide sequence set forth in SEQ ID NO: 3 as a template, and a pair of the primer Nos. 4 and 5 shown in Table 1, a GtTE gene consisting of the nucleotide sequence set forth in SEQ ID NO: 3 was prepared by PCR.

Moreover, using a plasmid vector pBluescriptII SK(−) (manufactured by Stratagene) as a template, and a pair of the primer Nos. 6 and 7 shown in Table 1, the pBluescriptII SK(−) was amplified by PCR. Then, the resultant template was subjected to digestion by restriction enzyme Dpnl (manufactured by TOYOBO) treatment.

A plasmid for GtTE gene expression GtTE_487 was constructed by purifying these two fragments using High Pure PCR Product Purification Kit (manufactured by Roche Applied Science Corporation), and then fusing the resultant material by using In-Fusion HD Cloning Kit (manufactured by Clontech, Inc.) to perform transformation into Escherichia coli DH5α strain Competent Cells (manufactured by Takara Bio), plasmid extraction, and confirmation of a nucleotide sequence of a cloning fragment according to an ordinary method.

In a similar manner, a plurality of plasmids for GtTE gene expression, in which an N-terminal region of the amino acid sequence set forth in SEQ ID NO: 1 was deleted at various lengths, were constructed.

PCR was carried out by using the plasmid GtTE_487 as a template, and a pair of any one of the primer Nos. 8 to 23 and the primer No. 6 shown in Table 1, and obtained gene fragments were purified and fused in a manner similar to the method described above, to construct plasmids for GtTE gene expression GtTE_497, GtTE_507, GtTE_517, GtTE_527, GtTE_537, GtTE_547, GtTE_557, GtTE_567, GtTE_577, GtTE_587, GtTE_597, GtTE_607, GtTE_608, GtTE_609, GtTE_610 and GtTE_611, respectively.

Herein, the plasmid GtTE_487 was constructed in the form of removing an amino acid sequence of the 1 st to 486th positions on an N-terminal side of an amino acid sequence set forth in SEQ ID NQ: 1, and had a nucleotide sequence of the 1st to 861st positions set forth in SEQ ID NO: 3 corresponding to a nucleotide sequence encoding the amino acid sequence of the 487th to 772nd positions set forth in SEQ ID NO: 1 and the termination codon as a GtTE gene. In a similar manner, the plasmid GtTE_497, the plasmid GtTE_507, the plasmid GtTE_517, the plasmid GtTE_527, the plasmid GtTE_537, the plasmid GtTE_547, the plasmid GtTE_557, the plasmid GtTE_567, the plasmid GtTE_577, the plasmid GtTE_587, the plasmid GtTE_597, the plasmid GtTE_607, the plasmid GtTE_608, the plasmid GtTE_609, the plasmid GtTE_610, and the plasmid GtTE_611, were constructed in the form of removing an amino acid sequence of the 1st to 498th positions, the 1st to 506th positions, the 1st to 516th positions, the 1st to 526th positions, the 1st to 536th positions, the 1st to 546th positions, the 1st to 556th positions, the 1st to 566th positions, the 1st to 576th positions, the 1st to 586th positions, the 1st to 596th positions, the 1st to 606th positions, the 1st to 607th positions, the 1st to 608th positions, the 1st to 609th positions, or the 1st to 610th positions, on an N-terminal side of an amino acid sequence set forth in SEQ ID NO: 1, respectively. Further, these plasmids were constructed in the form of expressing a protein fusing an amino acid sequence of the 1st to 29th positions on an N-terminal side of a LacZ protein derived from the plasmid vector pBluescriptII SK(−), to the upstream of the removed sites on an N-terminal side of the amino acid sequence set forth in SEQ ID NO: 1.

(3) Introduction of Plasmid for GtTE Gene Expression into Escherichia Coli

An Escherichia coli mutant strain, strain K27 (fadD88) (Overath et al, Eur. J. Biochem., vol 7, 559-574, 1989), was transformed by a competent cell transformation method, using the various plasmids for GtTE gene expression. The transformed strain K27 was inoculated in LB agar medium containing 50 μg/mL of Ampicillin sodium (Bacto Trypton 1%, Yeast Extract 0.5%, NaCl 1%, and Agar 1.5%), and was stand overnight at 30° C. The colony thus obtained was inoculated to 2 mL of Overnight Express Instant TB medium (Novagen) and was subjected to shaking culture at 30° C. After 24 hours cultivation, lipid components contained in the culture fluid were analyzed by the method described below. In addition, as a negative control, the Escherichia coli strain K27 transformed with the plasmid vector pBluescriptII SK(−) was also subjected to the same experiment.

(4) Extraction of Lipid from Culture Fluid and Analysis of Fatty Acids Contained Therein

To 1 mL of the culture fluid, 50 μL of 1 mg/mL 7-pentadecanone (methanol solution) as an internal standard was added, and then 0.5 mL of chloroform, 1 mL of methanol and 10 μL of 2N hydrochloric acid were further added to the cultured fluid. The mixture was vigorously stirred and then was left for 10 minutes or more. Further, 0.5 mL of chloroform and 0.5 mL of 1.5% KCl were added thereto. The mixture was stirred and centrifuged for 5 minutes at 3,000 rpm, and then the chloroform layer (lower layer) was collected with pasteur pipette.

A nitrogen gas was blown onto the resultant chloroform layer to be dried into solid, 0.7 mL of 0.5 N potassium hydroxide/methanol solution was added thereto, and the resultant mixture was kept warm at 80° C. for 30 minutes. Then, 1 mL of 14% methanol solution of boron trifluoride (manufactured by Sigma-Aldrich) was added to the sample, and the mixture was kept warm at 80° C. for 10 minutes. Thereafter, 1 mL of hexane and 1 mL of saturated saline were added thereto, and the mixture was vigorously stirred and then was left for 10 minutes or more at room temperature. Then, the hexane layer (upper layer) was collected to obtain fatty acid methyl esters.

Under the measuring conditions as follows, the obtained fatty acid methyl esters were provided for gas chromatographic analysis.

### <Gas Chromatography Conditions>

Capillary column: DB-1 MS (30 m×200 μm×0.25 μm, manufactured by J&W Scientific)
Mobile phase: high purity helium
Flow rate inside the column: 1.0 mL/min
Temperature rise program: 100° C. (for 1 min.)→10° C./min→300° C. (for 5 min.)
Equilibration time: for 1 min.
Injection port: split infection (split ratio: 100:1)
Pressure: 14.49 psi, 104 mL/min

### Amount of Injection: 1 μL

Cleaning vial: methanol/chloroform
Detector temperature: 300° C.

Further, the fatty acid methyl esters were identified by providing the identical sample for a gas chromatography-mass spectrometry analysis under identical conditions described above.

Amounts of the fatty acid methyl esters were quantitatively determined based on the peak areas of waveform data obtained by the above gas chromatographic analysis. The peak area corresponding to each of the fatty acid methyl esters was compared with that of 7-pentadecanone as the internal standard, and carried out corrections between the samples, and then the amount of each of the fatty acids per liter of the culture fluid was calculated. Further, the total amount of the fatty acids was calculated by summing the amounts of each of the fatty acids thus obtained, and ratio of amounts of each of the fatty acids in the total amount of the fatty acids was calculated.

The results are shown in Table 3. Herein, in Table below, “TFA” presents a total amount of fatty acids, and “Fatty Acid Composition (% TFA)” presents a ratio of a weight of each fatty acid relative to a weight of the total fatty acid (weight percent). Moreover, description of “Cx:y” represents a fatty acid having “x” as the number of carbon atoms, and “y” as the number of double bonds, and the expressions “C17:0Δ” and “C19:0Δ” designate cis-9,10-Methylen-hexadecanoic acid and cis-t11,12-Methylen-octadecanoic acid, respectively.

TABLE 3

| Introduced | TFA | Fatty acid composition (% TFA) | | | | | | | | | |
|---|---|---|---|---|---|---|---|---|---|---|---|
| plasmid | (mg/L) | C10:0 | C12:1 | C12:0 | C14:1 | C14:0 | C16:1 | C16:0 | C17:0Δ | C18.1 | C19:0Δ |
| pBS | 171.0 | 0.0 | 0.0 | 0.0 | 0.0 | 5.1 | 1.7 | 48.6 | 28.2 | 3.4 | 12.9 |
| GtTE_487 | 310.6 | 2.0 | 1.5 | 6.0 | 2.3 | 15.1 | 3.6 | 36.0 | 20.0 | 3.3 | 10.1 |
| GtTE_497 | 308.9 | 2.0 | 1.5 | 6.0 | 2.4 | 13.7 | 4.0 | 35.4 | 20.4 | 4.1 | 10.5 |
| GtTE_507 | 298.7 | 2.3 | 1.6 | 6.2 | 2.5 | 13.2 | 4.0 | 35.0 | 20.3 | 3.9 | 11.1 |
| GtTE_517 | 321.5 | 2.7 | 2.0 | 6.9 | 3.0 | 14.6 | 4.9 | 32.9 | 18.6 | 4.4 | 9.9 |
| GtTE_527 | 274.1 | 1.9 | 1.5 | 5.1 | 2.4 | 12.3 | 5.8 | 35.6 | 19.4 | 6.5 | 9.5 |
| GtTE_537 | 253.2 | 0.0 | 1.7 | 4.9 | 2.6 | 10.3 | 8.7 | 36.3 | 17.8 | 10.5 | 7.2 |
| GtTE_547 | 307.2 | 2.2 | 1.5 | 6.0 | 2.4 | 13.7 | 4.2 | 35.1 | 20.0 | 4.5 | 10.3 |
| GtTE_557 | 249.7 | 0.0 | 0.7 | 3.8 | 1.5 | 11.4 | 2.9 | 40.6 | 23.8 | 3.6 | 11.6 |
| GtTE_567 | 287.4 | 0.0 | 1.9 | 6.9 | 2.8 | 16.6 | 4.7 | 35.0 | 18.7 | 4.1 | 9.2 |
| GtTE_577 | 301.7 | 2.3 | 1.6 | 6.0 | 2.5 | 15.0 | 4.1 | 35.4 | 18.8 | 4.4 | 9.9 |
| GtTE_587 | 460.9 | 4.0 | 4.5 | 8.9 | 5.7 | 17.7 | 11.1 | 25.0 | 10.8 | 8.9 | 3.3 |
| GtTE_597 | 694.5 | 4.7 | 7.8 | 10.3 | 8.9 | 19.2 | 17.5 | 17.4 | 5.9 | 7.5 | 0.8 |
| GtTE_607 | 672.0 | 4.5 | 7.2 | 10.3 | 8.4 | 19.5 | 16.0 | 18.5 | 6.5 | 7.4 | 1.7 |
| GtTE_608 | 305.1 | 4.1 | 3.5 | 8.7 | 4.2 | 17.0 | 6.9 | 29.0 | 14.8 | 5.1 | 6.7 |
| GtTE_609 | 419.4 | 4.4 | 4.7 | 10.5 | 5.5 | 18.3 | 9.2 | 25.0 | 10.6 | 7.0 | 4.7 |
| GtTE_610 | 262.4 | 2.6 | 2.3 | 5.7 | 3.1 | 12.1 | 9.0 | 33.6 | 15.7 | 10.1 | 5.8 |
| GtTE_611 | 212.2 | 0.0 | 1.1 | 4.4 | 1.9 | 9.8 | 3.9 | 39.4 | 24.1 | 5.6 | 9.8 |

As shown in Table 3, in the strain having the introduced any one of the various plasmids for GtTE gene expression, a ratio of each of the C12:1, C12:0, C14:1, C14:0, and C16:1 fatty adds in the total fatty add significantly increased in comparison with the strain having the introduced the negative control plasmid vector pBluescriptII SK(−) (“pBS” in Table). In particular, a ratio of C14 fatty acids (C14:1 and C14:0 fatty acids) extremely increased. Further, in the strain having the introduced these plasmids for GtTE gene expression, the total amount of fatty acids (TFA) also increased. In particular, in the strain having the introduced the plasmid GtTE_587, GtTE_597, GtTE_607, GtTE_608 or GtTE_609, increase of C12:1, C12:0, C14:1, C14:0 and C16:1 fatty acids, and increase of the total amount of fatty acids were significant.

From these results, it was confirmed that the proteins encoding the gene introduced into the various plasmids for GtTE gene expression had acyl-ACP thioesterase activity. Moreover, these proteins extremely increased a ratio and productivity of the C12 and C14 fatty acids. Therefore, it was considered that these proteins are acyl-ACP thioesterases having high specificity to the C12 and C14 fatty acids, particularly C14 fatty acids.

From the results described above, it is recognized that the protein having the region of at least 611th to 772nd positions in the amino acid sequence set forth in SEQ ID NO: 1 designates acyl-ACP thioesterase activity.

### Examples 2

### Transformation of Nannochloropsis oculata by GtTE Gene, and Producing Lipid by Transformant

### (1) Construction of Plasmid for Zeocin Resistance Gene Expression

A zeocin resistance gene (SEQ ID NO: 24), and a tubulin promoter sequence (SEQ ID NO: 25) derived from Nannochloropsis gaditana strain CCMP 526 described in a literature (Randor Radakovits, et al., Nature Communications, DOI: 10.038/ncomms1688, 2012) were artificially synthesized. Using the thus-synthesized DNA fragments as a template, and a pair of the primer Nos. 26 and 27, and a pair of the primer Nos. 28 and 29 shown in Table 2, PCR was carried out, to amplify the zeocin resistance gene and the tubulin promoter sequence, respectively.

Further, using a genome of Nannochloropsis oculata strain NIES2145 as a template, and a pair of the primer Nos. 30 and 31 shown in Table 2, PCR was carried out to amplify the heat shock protein terminator sequence (SEQ ID NO: 32).

Furthermore, using a plasmid vector pUC19 (manufactured by Takara Bio) as a template, and a pair of the primer Nos. 33 and 34 shown in Table 2, PCR was carried out to amplify the plasmid vector pUC19.

These four amplified fragments were treated by restriction enzyme Dpnl (manufactured by TOYOBO) respectively, and were purified using a High Pure PCR Product Purification Kit (manufactured by Roche Applied Science). Then, obtained four fragments were fused using an in-Fusion HD Cloning Kit (manufactured by Clontech) to construct a plasmid for zeocin resistance gene expression.

Herein, the plasmid consisted of the pUC19 vector sequence and an insert sequence in which the tubulin promoter sequence, the zeocin resistance gene and the heat shock protein terminator sequence were linked in this order.

### (2) Construction of Plasmid for GtTE Gene Expression

Using the GtTE gene artificially synthesized in Example 1 as a template, and a pair of any one of the primer Nos. 35 to 39 and the primer No. 40 shown in Table 2, PCR was carried out to prepare GtTE gene fragments, in which 5′ side of the nucleotide sequence set forth in SEQ ID NO: 3 was deleted at various lengths.

Further, using a genome of Nannochloropsis oculata strain NIES2145 as a template, and a pair of the primer Nos. 41 and 42, a pair of the primer Nos. 43 and 44, and a pair of the primer Nos. 45 and 46 shown in Table 2, respectively, PCR was carried out to prepare the LDSP promoter sequence (SEQ ID NO: 47), the VCP1 chloroplast transit signal sequence (SEQ ID NO: 48), and the VCP1 terminator sequence (SEQ ID NO: 49).

Furthermore, using the above-described plasmid for zeocin resistance gene expression as a template, and a pair of the primer Nos. 50 and 34 shown in Table 2, PCR was carried out to amplify a fragment containing the cassette for zeocin resistance gene expression (the tubulin promoter sequence, the zeocin resistance gene, and the heat shock protein terminator sequence) and the pUC19 sequence.

Respective GtTE gene fragments, in which 5′ side of the nucleotide sequence set forth in SEQ ID NO: 3 was deleted at various lengths, the amplified fragments of the LDSP promoter, the VCP1 chloroplast transit signal and the VCP1 terminator, and the amplified fragments containing the cassette for zeocin resistance gene expression and the pUC19 sequence were fused by a method in a manner similar to described above, to construct plasmids for GtTE gene expression GtTE_488-Nanno, GtTE_527-Nanno, GtTE_587-Nanno, GtTE_597-Nanno and GtTE_607-Nanno, respectively.

Herein, these plasmids consisted of the pUC19 vector sequence and an insert sequence in which the LDSP promoter sequence, the GtTE gene in which the VCP1 chloroplast transit signal was linked to the 5′-terminal side of the nucleotide sequence encoding an amino acid sequence of the 488th to 772nd positions, the 527th to 772nd positions, the 587th to 772nd positions, the 597th to 772nd positions, or the 607th to 772nd positions set forth in SEQ ID NO: 1, the VCP1 terminator sequence, the tubulin promoter sequence, the zeocin resistance gene and the heat shock protein terminator sequence were linked in this order.

(3) Introduction of Cassette for GtTE Gene Expression into Nannochloropsis Oculata

Using the above-described plasmids for GtTE gene expression (GtTE_488-Nanno, GtTE_527-Nanno, GtTE_587Nanno, GtTE_597-Nanno and GtTE_607-Nanno) as a template, respectively, and a pair of the primer Nos. 41 and 31 shown in Table 2, PCR was carried out to amplify cassettes for GtTE gene expression (a DNA fragment containing the LDSP promoter sequence, the VCP1 chloroplast transit signal, the GtTE gene in the form of removing the nucleotide sequence encoding an amino acid sequence of the 1st to 487th positions, the 1st to 526th positions, the 1st to 586th positions, the 1st to 596th positions, or the 1st to 606th positions on an N-terminal side of an amino acid sequence set forth in SEQ ID NO: 1, the VCP1 terminator sequence, the tubulin promoter sequence, the zeocin resistance gene, and the heat shock protein terminator sequence), respectively.

The amplified fragments were purified using High Pure PCR Product Purification Kit (manufactured by Roche Applied Science), respectively. Herein, sterilized water was used for elution upon purification without using an elution buffer included in the kit.

About 1×10⁹ cells of Nannochloropsis oculata strain NIES2145 were washed with 384 mM sorbitol solution to completely remove a salt, and the resultant was used as a host cell of transformation. The cassette for GtTE gene expression as amplified above was mixed by about 500 ng for each with the host cell, and electroporation was carried out under conditions of 50 μF, 500Ω and 2,200 v/2 mm.

After 24 hours recovery cultivation in f/2 liquid medium (75 mg of NaNO₃, 6 mg of NaH₂PO₄.2H₂O, 0.5 μg of vitamin B12, 0.5 μg of biotin, 100 μg of thiamine, 10 mg of Na₂SiO₃.9H₂O, 4.4 mg of Na₂EDTA.2H₂O, 3.16 mg of FeCl₃.6H₂O, 12 μg of FeCl₃.6H₂O, 21 μg of ZnSO₄.7H₂O, 180 μg of MnCl₂.4H₂O, 7 μg of CuSO₄.5H₂O, 7 μg of Na₂MoO₄.2H₂O/artificial sea water 1 L), the resultant material was inoculated in f/2 agar medium containing 2 μg/mL of zeocin, sod cultured for two to three weeks under 12 h/12 h light-dark conditions at 25° C. under an atmosphere of 0.3% CO₂. A transformant containing the cassette for GtTE gene expression was selected from the resultant colonies by a PCR method.

(4) Extraction of Lipid from Culture Fluid and Analysis of Fatty Aids Contained Therein

The selected strain was inoculated to 20 mL of medium in which a nitrogen concentration in the f/2 medium was reinforced 15 times, and a phosphorus concentration therein was reinforced 5 times (hereinafter, referred to as “N15P5 medium”), and subjected to shaking culture for four weeks under the 12 h/12 h light-dark conditions at 25° C. under the atmosphere of 0.3% CO₂, to prepare preculture fluid. Then, 2 mL of the preculture fluid was inoculated to 18 mL of the N15P5 medium, and subjected to shaking culture for three weeks under the 12 h/12 h light-dark conditions at 25° C. under the atmosphere of 0.3% CO₂.

in addition, as a negative control, an experiment was also conducted on the wild type strain, Nannochloropsis oculata strain NIES2145.

To 1 mL of the culture fluid, 50 μL of 1 mg/mL 7-pentadecanone (methanol solution) as an internal standard was added, and then 0.5 mL of chloroform and 1 mL of methanol were further added. The mixture was vigorously stirred and then was left for 10 minutes. Further, 0.5 mL of chloroform and 0.5 mL of 1.5% KCl were added thereto. The mixture was stirred and centrifuged for 5 minutes at 3,000 rpm, and then the chloroform layer (lower layer) was collected with pasteur pipette.

A nitrogen gas was blown onto the resultant chloroform layer to be dried into solid, 0.7 mL of 0.5 N potassium hydroxide/methanol solution was added thereto, and the resultant mixture was kept warm at 80° C. for 30 minutes. Then, 1 mL of 14% methanol solution of boron trifluoride (manufactured by Sigma-Aldrich) was added to the sample, and the mixture was kept warm at 80° C. for 10 minutes. Thereafter 0.5 mL of hexane and 1 mL of saturated saline were added thereto, and the mixture was vigorously stirred and then was left for 10 minutes at room temperature. Then, the hexane layer (upper layer) was collected to obtain fatty acid methyl esters.

Under the measuring conditions as follows, the obtained fatty acid methyl esters were provided for gas chromatographic analysis.

### <Gas Chromatography Conditions>

Analysis apparatus: 7890A (manufactured by Agilent Technologies)
Capillary column: DB-WAX (10 m×100 μm×0.10 μm, manufactured by J&W Scientific)
Mobile phase: high purity helium
Oven temperature: maintained for 0.5 min. at 100° C.→100° C. to 250° C. (temperature increase at 20° C./min)→maintained for 3 min. at 250° C. (post run: 1 min.)
Injection port, temperature: 300° C.
Injection method: split injection (split ratio: 50:1)
Amount of injection: 5 μL
Cleaning vial: methanol
Detection method: FID
Detector temperature: 350° C.

The fatty acid methyl esters were identified and quantitatively determined according to a method similar to Example 1. Table 4 shows the results.

TABLE 4

| | | | | | | | | | | | | | | | Contents |
|---|---|---|---|---|---|---|---|---|---|---|---|---|---|---|---|
| | | | | | | | | | | | | | | | of C8 |
| | | | | | | | | | | | | | | | to C14 |
| | Fatty acid composition (% TFA) | | | | | | | | | | | | | | fatty |
| Introduced | C8: | C10: | C12: | C14: | C16: | C16: | C18: | C18: | C18: | C18: | C20: | C20: | C20: | C20: | acid |
| DNA | 0 | 0 | 0 | 0 | 0 | 1 | 0 | 1 | 2 | 3 | 0 | 3 | 4 | 5 | (mg/L) |
| WT | 0.0 | 58.9 | 0.2 | 4.0 | 34.1 | 30.4 | 1.4 | 16.1 | 1.5 | 0.5 | 0.1 | 0.2 | 2.1 | 9.4 | 58.9 |
| GtTE_488-Nanno | 0.0 | 79.5 | 1.6 | 5.0 | 25.2 | 36.2 | 1.3 | 11.7 | 1.6 | 0.5 | 0.2 | 0.3 | 2.6 | 13.3 | 79.6 |
| GtTE_527-Nanno | 0.0 | 85.0 | 0.8 | 4.7 | 30.6 | 34.3 | 1.2 | 12.7 | 1.6 | 0.4 | 0.2 | 0.2 | 2.3 | 10.7 | 85.0 |
| GtTE_587-Nanno | 0.2 | 199.2 | 4.2 | 6.4 | 20.0 | 37.2 | 0.9 | 10.9 | 1.5 | 0.7 | 0.2 | 0.3 | 3.4 | 11.8 | 199.2 |
| GtTE_597-Nanno | 0.3 | 165.7 | 3.9 | 5.8 | 16.3 | 37.0 | 0.7 | 10.8 | 1.6 | 0.6 | 0.1 | 0.4 | 3.5 | 15.9 | 165.7 |

As shown in Table 4, in any of the Nannochloropsis transformants having the introduced cassette for GtTE gene expression (“GtTE_488-Nanno”, “GtTE_527-Nanno”, “GtTE_587-Nanno”, “GtTE_597-Nanno” and “GtTE_607-Nanno”, in Table 4), a ratio of each of the C10:0, C12:0 and C14:0 fatty acids increased in comparison with the wild type strain (“WT” in Table 4). Further, in two kinds of transformants among these (GtTE_587-Nanno and GtTE_597-Nanno), C8:0 fatty acid, which was not detected at all in the wild type strain, was detected. Furthermore, in all of the transformants, productivity of medium chain fatty acids (C8 to C14 fatty acids) increased in comparison with the wild type strain.

As described above, the transformant in which productivity of the medium chain fatty acids and the productivity of the total fatty acids to be produced are improved can be prepared by promoting the expression of the acyl-ACP thioesterase gene as specified in the present invention. Further, productivity of the medium chain fatty acids can be improved by culturing this transformant.

Having described our invention as related to the present embodiments. It is our intention that the invention not be limited by any of the details of the description, unless otherwise specified, but rather be construed broadly within its spirit and scope as set out in the accompanying claims.

This application claims priority on Patent Application No. 2014-246573 filed in Japan on Dec. 5, 2014, which is entirely herein incorporated by reference.

What is claimed is:

1. A method of producing a lipid, comprising the steps of:
culturing a transformant in which a gene encoding any one of the following proteins (A) to (C) is introduced into a host, and
collecting a lipid from the cultured product:
(A) a protein consisting of the amino acid sequence of the 611th to 772nd positions set forth in SEQ ID NO: 1;
(B) a protein consisting of an amino acid sequence having 80% or more identity with the amino acid sequence of the 611th to 772nd positions set forth in SEQ ID NO: 1, and having acyl-ACP thioesterase activity; and
(C) a protein containing the amino acid sequence of the protein (A) or (B), and having acyl-ACP thioesterase activity.

2. (canceled)

3. A method of modifying the composition of a lipid, comprising the steps of:
introducing a gene encoding any one of the following proteins (A) to (C) into a host, and thereby obtaining a transformant, and
enhancing productivity of medium chain fatty acids or a lipid containing the fatty acids as components produced in a cell of the transformant, to modify the composition of fatty acids or a lipid in all fatty acids or all lipids to be produced:
(A) a protein consisting of the amino acid sequence of the 611th to 772nd positions set forth in SEQ ID NO: 1;
(B) a protein consisting of an amino acid sequence having 80% or more identity with the amino acid sequence of the 611th to 772nd positions set forth in SEQ ID NO: 1, and having acyl-ACP thioesterase activity; and
(C) a protein containing the amino acid sequence of the protein (A) or (B), and having acyl-ACP thioesterase activity.

4. The method according to claim 1, wherein the protein (C) is any one of the following proteins (C1) to (C20):
(C1) a protein consisting of the amino acid sequence of the 1st to 772nd positions set forth in SEQ ID NO: 1;
(C2) a protein consisting of the amino acid sequence of the 487th to 772nd positions set forth in SEQ ID NO: 1;
(C3) a protein consisting of the amino acid sequence of the 488th to 772nd positions set forth in SEQ ID NO: 1;
(C4) a protein consisting of the amino acid sequence of the 497th to 772nd positions set forth in SEQ ID NO: 1;
(C5) a protein consisting of the amino acid sequence of the 507th to 772nd positions set forth in SEQ ID NO: 1;
(C6) a protein consisting of the amino acid sequence of the 517th to 772nd positions set forth in SEQ ID NO: 1;
(C7) a protein consisting of the amino acid sequence of the 527th to 772nd positions set forth in SEQ ID NO: 1;
(C8) a protein consisting of the amino acid sequence of the 537th to 772nd positions set forth in SEQ ID NO: 1;
(C9) a protein consisting of the amino acid sequence of the 547th to 772nd positions set forth in SEQ ID NO: 1;
(C10) a protein consisting of the amino acid sequence of the 557th to 772nd positions set forth in SEQ ID NO: 1;
(C11) a protein consisting of the amino acid sequence of the 567th to 772nd positions set forth in SEQ ID NO: 1;
(C12) a protein consisting of the amino acid sequence of the 577th to 772nd positions set forth in SEQ ID NO: 1;
(C13) a protein consisting of the amino acid sequence of the 587th to 772nd positions set forth in SEQ ID NO: 1;
(C14) a protein consisting of the amino acid sequence of the 597th to 772nd positions set forth in SEQ ID NO: 1;
(C15) a protein consisting of the amino acid sequence of the 607th to 772nd positions set forth in SEQ ID NO: 1;
(C16) a protein consisting of the amino acid sequence of the 608th to 772nd positions set forth in SEQ ID NO: 1;
(C17) a protein consisting of the amino acid sequence of the 609th to 772nd positions set forth in SEQ ID NO: 1;
(C18) a protein consisting of the amino acid sequence of the 610th to 772nd positions set forth in SEQ ID NO: 1;
(C19) a protein consisting of an amino acid sequence having 80% or more identity with the amino acid sequence of any one of the proteins (C1) to (C18), and having acyl-ACP thioesterase activity; and
(C20) a protein consisting of an amino acid sequence in which 1 or several amino acids are deleted, substituted, inserted or added to the amino acid sequence of any one of the proteins (C1) to (C18), and having acyl-ACP thioesterase activity.

5. The method according to claim 3, wherein the protein (C) is any one of the following proteins (C1) to C20):
(C1) a protein consisting of the amino acid sequence of the 1st to 772nd positions set forth in SEQ ID NO: 1;
(C2) a protein consisting of the amino acid sequence of the 487th to 772nd positions set forth in SEQ ID NO: 1;
(C3) a protein consisting of the amino acid sequence of the 488th to 772nd positions set forth in SEQ ID NO: 1;
(C4) a protein consisting of the amino acid sequence of the 497th to 772nd positions set forth in SEQ ID NO: 1;
(C5) a protein consisting of the amino acid sequence of the 507th to 772nd positions set forth in SEQ ID NO: 1;
(C6) a protein consisting of the amino acid sequence of the 517th to 772nd positions set forth in SEQ ID NO: 1;
(C7) a protein consisting of the amino acid sequence of the 527th to 772nd positions set forth in SEQ ID NO: 1;
(C8) a protein consisting of the amino acid sequence of the 537th to 772nd positions set forth in SEQ ID NO: 1;
(C9) a protein consisting of the amino acid sequence of the 547th to 772nd positions set forth in SEQ ID NO: 1;
(C10) a protein consisting of the amino acid sequence of the 557th to 772nd positions set forth in SEQ ID NO: 1;
(C11) a protein consisting of the amino acid sequence of the 567th to 772nd positions set forth in SEQ ID NO: 1;
(C12) a protein consisting of the amino acid sequence of the 577th to 772nd positions set forth in SEQ ID NO: 1;
(C13) a protein consisting of the amino acid sequence of the 587th to 772nd positions set forth in SEQ ID NP: 1;
(C14) a protein consisting of the amino acid sequence of the 597th to 772nd positions set forth in SEQ ID NO: 1;
(C15) a protein consisting of the amino acid sequence of the 607th to 772nd positions set forth in SEQ ID NO: 1;
(C16) a protein consisting of the amino acid sequence of the 608th to 772nd positions set forth in SEQ ID NO: 1;
(C17) a protein consisting of the amino acid sequence of the 609th to 772nd positions set forth in SEQ ID NO: 1;
(C18) a protein consisting of the amino acid sequence of the 610th to 772nd positions set forth in SEQ ID NO: 1;
(C19) a protein consisting of an amino acid sequence having 80% or more identity with the amino acid sequence of any one of the proteins (C1) to (C18) and having acyl-ACP thioesterase activity; and
(C20) a protein consisting of an amino acid sequence in which 1 or several amino acids are deleted, substituted, inserted or added to the amino acid sequence of any one of the proteins (C1) to (C18) and having acyl-ACP thioesterase activity.

6. The method according to claim 1, wherein the host is Escherichia coli.

7. The method according to claim 3, wherein the host is Escherichia coli.

8. The method according to claim 1, wherein the host is a microalga.

9. The method according to claim 8, wherein the microalga is an alga belonging to the genus Nannochloropsis.

10. The method according to claim 1, wherein the lipid contains a fatty acid having 12 carbon atoms or a fatty acid ester compound thereof.

11. The method according to claim 3, wherein the host is a microalga.

12. The method protein according to claim 11, wherein the microalga is an alga belonging to the genus Nannochloropsis.

13. The method according to claim 3, wherein the lipid contains a fatty acid having 12 carbon atoms or a fatty acid ester compound thereof.

14.-15. (canceled)

16. A transformant, which is obtained by introducing a gene encoding any one of the following proteins (A) to (C) into a host:
(A) a protein consisting of the amino acid sequence of the 611th to 772nd positions set forth in SEQ ID NO: 1;
(B) a protein consisting of an amino acid sequence having 80% or more identity with the amino acid sequence of the 611th to 772nd positions set forth in SEQ ID NO: 1, and having acyl-ACP thioesterase activity; and
(C) a protein containing the amino acid sequence of the protein (A) or (B), and having acyl-ACP thioesterase activity.

17. The transformant according to claim 16, wherein the protein (C) is any one of the following proteins (C1) to (C20):
(C1) a protein consisting of the amino acid sequence of the 1st to 772nd positions set forth in SEQ ID NO: 1;
(C2) a protein consisting of the amino acid sequence of the 487th to 772nd positions set forth in SEQ ID NO: 1;
(C3) a protein consisting of the amino acid sequence of the 488th to 772nd positions set forth in SEQ ID NO: 1;
(C4) a protein consisting of the amino acid sequence of the 497th to 772nd positions set forth in SEQ ID NO: 1;
(C5) a protein consisting of the amino acid sequence of the 507th to 772nd positions set forth in SEQ ID NO: 1;
(C6) a protein consisting of the amino acid sequence of the 517th to 772nd positions set forth in SEQ ID NO: 1;
(C7) a protein consisting of the amino acid sequence of the 527th to 772nd positions set forth in SEQ ID NO: 1;
(C8) a protein consisting of the amino acid sequence of the 537th to 772nd positions set forth in SEQ ID NO: 1;
(C9) a protein consisting of the amino acid sequence of the 547th to 772nd positions set forth in SEQ ID NO: 1;
(C10) a protein consisting of the amino acid sequence of the 557th to 772nd positions set forth in SEQ ID NO: 1;
(C11) a protein consisting of the amino acid sequence of the 567th to 772nd positions set forth in SEQ ID NO: 1;
(C12) a protein consisting of the amino acid sequence of the 577th to 772nd positions set forth in SEQ ID NO: 1;
(C13) a protein consisting of the amino acid sequence of the 587th to 772nd positions set forth in SEQ ID NO: 1;
(C14) a protein consisting of the amino acid sequence of the 597th to 772nd positions set forth in SEQ ID NO: 1;
(C15) a protein consisting of the amino acid sequence of the 607th to 772nd positions set forth in SEQ ID NO: 1;
(C16) a protein consisting of the amino acid sequence of the 608th to 772nd positions set forth in SEQ ID NO: 1;
(C17) a protein consisting of the amino acid sequence of the 609th to 772nd positions set forth in SEQ ID NO: 1;
(C18) protein consisting of the amino acid sequence of the 610th to 772nd positions set forth in SEQ ID NO: 1;
(C19) a protein consisting of an amino acid sequence having 80% or more identity with the amino acid sequence of any one of the proteins (C1) to (C18), and having acyl-ACP thioesterase activity; and
(C20) a protein consisting of an amino acid sequence in which 1 or several amino acids are deleted, substituted, inserted or added to the amino acid sequence of any one of the proteins (C1) to (C18) and having acyl-ACP thioesterase activity.

18. The transformant according to claim 16, wherein the host is Escherichia coli.

19. The transformant according to claim 16, wherein the host is a microalga.

20. The transformant according to claim 19, wherein the microalga is an alga belonging to the genus Nannochloropsis.
